SEQUENCE LISTING (12 sequences)

SEQ ID NO: 1 - DNA, 4352 bp, Homo sapiens
atatctatagttacaatttgaggttcaggtaaataaactctagatcttatagaactgcggtaaggtaggatagggagactccttcgactttctctgtttatttgtctctatttttaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 2 - DNA, 27 bp, Artificial Sequence
  gRNAHu1 target sequence
taaagatccagacctgccccgaggaat

SEQ ID NO: 3 - DNA, 27 bp, Artificial Sequence
  gRNAHu2 target sequence
cttataaggataccaactggattggat

SEQ ID NO: 4 - DNA, 3156 bp, Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 5 - DNA, 27 bp, Artificial Sequence
  synthetic polynucleotide
attcctcggggcaggtctggatcttta

SEQ ID NO: 6 - DNA, 27 bp, Artificial Sequence
  synthetic polynucleotide
atccaatccagttggtatccttataag

SEQ ID NO: 7 - protein, 605 aa, Artificial Sequence
  Amino acid sequence of ZFN16C
MPAAKRVKLDYACPVESCDRRFSTSGHLVRHIRIHTGEKPFQCRICMRNFSRDSHLSRHIRTHTGEKPFACDICGRKFATSANLSRHTKIHTGQKDQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQAREMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGATNFSLLKQAGDVEENPGPPAAKRVKLDYACPVESCDRRFSTSANLSRHIRIHTGEKPFQCRICMRNFSRNDALTEHIRTHTGEKPFACDICGRKFAQNSTLTEHTKIHTGQKDQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQAREMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 8 - DNA, 2256 bp, Artificial Sequence
  Nucleotide sequence of ZFN16C
atgcccgccgccaagcgggtgaagctggactacgcctgcccagtggaaagttgtgaccggcggttcagccgcgacagccacctgtcccgccacatccggatccatacaggcgagaaacctttccagtgcagaatctgcatgagaaatttcagccagagcagctctctggtgcggcacatcagaacccacacaggagagaagcctttcgcctgcgatatctgtggaagaaagttcgccacctccggacaccttgtgagacatacaaaaatccacacaggctctgagagaccttatgcctgccctgtggagtcttgtgacagacggttcagcagagatagccacctgagcagacatatcagaatccatacaggcgagaagccctttcagtgccggatctgcatgagaaacttcagtacaagcgccaatctgagcagacacatccggacccacaccggacagaaagaccagctggtgaagtctgagctggaagaaaagaagagcgaactgagacacaagctgaagtacgtgccacacgagtacatcgagctgatcgaaatcgcccggaacagcacccaggatcggattctggaaatgaaggtgatggaattcttcatgaaagtgtatggctacagaggcaaacacctgggcggcagcagaaaacctgatggcgccatctacaccgttggatctcctatcgactacggcgtgattgtcgacacaaaggcctacagcggcgggtacaacctgcctatcggccaggccagagagatgcagcggtacgtggaggaaaaccagaccagaaacaagcacatcaaccccaacgagtggtggaaggtgtatcctagctccgtgaccgagttcaagttcctgttcgtgtccggccacttcaagggcaactacaaggctcagctaacccgcctcaaccacatcaccaattgcaatggcgctgttctgtctgtggaagagctgctgatcggcggcgagatgattaaggccggcaccctgaccctggaggaagtgagaagaaagtttaacaacggcgaaatcaacttcggctctggcgccaccaacttttctctgctgaaacaggccggcgacgtggaggagaaccccggccctcctgccgctaaacgggtgaaactggattacgcgtgtcccgtggaatcctgcgatagaagattctctagaagcgaccacctgagcagacacatccggatccacaccggcgaaaagcccttccagtgccggatctgcatgcggaacttcagcaccctgagcctgcacaccgaacacatccggacccacacaggcgagaagccattcgcctgtgatatctgtggcaggaagttcgcccagaacagcaccctgaccgagcacaccaagatccacaccggcagcgagcggccttacgcctgccctgtcgagagctgcgatcggcgattttccaccagcgccaacctcagcaggcatatcagaatccacacaggcgagaaaccttttcagtgtagaatctgcatgagaaacttcagcaggaacgacgccctgacagagcacatcagaacccacaccggagaaaagccgttcgcctgcgacatctgcggtagaaaattcgctcaaaatagcacactgacagagcacaccaagatccacactggacaaaaggaccagctggtcaagagcgagctcgaagagaagaaaagcgagctgagacataagctgaagtatgtgcctcacgagtacatcgagctgatcgagatcgctagaaacagcacccaggacagaatcctggagatgaaggtgatggaatttttcatgaaggtgtacggctaccggggcaagcacctgggcggatctcggaaacctgacggcgccatctacaccgtgggctccccaattgactacggcgtgatcgtggacaccaaggcttacagcggcggatacaacctgcccatcggccaggctagagagatgcagagatacgtggaagagaatcagacaagaaacaagcacatcaaccctaatgagtggtggaaggtgtaccccagcagcgtgacagaattcaagttcctgttcgtgtctggccactttaagggcaattacaaggcccaactgaccagactgaaccacatcaccaactgcaacggcgccgtgctgagcgtggaagagctgctgattggaggagagatgattaaggccggcacactgaccctggaagaagtgcggagaaagttcaacaacggcgaaatcaacttctaa

SEQ ID NO: 9 - DNA, 20 bp, Artificial Sequence
  synthetic polynucleotide
gaaaggaaacagaggcacac

SEQ ID NO: 10 - DNA, 21 bp, Artificial Sequence
  synthetic polynucleotide
agataaagatccagacctgcc

SEQ ID NO: 11 - DNA, 21 bp, Artificial Sequence
  synthetic polynucleotide
agaaaggaaacagaggcacac

SEQ ID NO: 12 - DNA, 20 bp, Artificial Sequence
  synthetic polynucleotide
ttcacgcataccccaggaac

(19) United States
(12) Patent Application Publication
(10) Pub. No.: US 2024/0425877 A1
(43) Pub. Date: Dec. 26, 2024

(54) ABCA4 GENOME EDITING

(71) Applicants: UCL BUSINESS LTD, London (GB); KING'S COLLEGE LONDON, London (GB)

(72) Inventors: Robin ALI, London (GB); Alexander SMITH, London (GB); Leticia Agundez CORTES, London (GB); Anai Gonzalez CORDERO, London (GB)

(21) Appl. No.: 18/289,524

(22) PCT Filed: May 6, 2022
(86) PCT No.: PCT/GB2022/051163

§ 371 Date: Nov. 3, 2023

(30) Foreign Application Priority Data
May 7, 2021 (GB) 2106516.4

(51) Int. Cl. C12N 15/86; A61K 48/00; C12N 9/22; C12N 15/11

(52) U.S. Cl. CPC C12N 15/86; C12N 9/22; C12N 15/11; A61K 48/00; C12N 2310/20; C12N 2750/14143; C12N 2800/80

(57) ABSTRACT

The present invention relates to a vector system for the in situ correction of the ABCA4 gene, and medical uses thereof.

### RELATED INFORMATION PARAGRAPH

This application is a 35 U.S.C. § 371 national stage filing of International Application No. PCT/GB2022/051163, filed May 6, 2022, which claims priority to GB Application No. 2106516.4, filed May 7, 2021. The contents of the aforementioned applications are hereby incorporated by reference in their entirety.

### FIELD OF THE INVENTION

The present invention relates to a vector system for the in situ correction of the ABCA4 gene, and medical uses thereof.

### BACKGROUND OF THE INVENTION

Stargardt disease (STGD) is an autosomal recessive inherited retinal disorder (TRD) caused by biallelic mutations in the ABCA4 gene. ABCA4-Stargardt disease is the most common form of hereditary macular dystrophy with a prevalence of around 1 in 10,000. More than 1000 different mutations have been classified as pathogenic. In many cases, deep-intronic mutations have been shown to be causative as they result in mRNA mis-splicing. These mRNA-splicing defects lead to either pseudoexon formation or exon skipping which in turn disrupt the normal sequence of codons by frameshift mutations or premature stop codon insertion.

The ABCA4 protein is an ATP-binding cassette (ABC) transporter in photoreceptor outer segments that functions in the visual cycle. More specifically, it is an N-retinylidene-phosphatidylethanolamine and phosphatidylethanolamine importer, the only known importer among mammalian ABC transporters. ABCA4 dysfunction results in accumulation of all-trans and 11-cis retinoids in photoreceptors (PRs), formation of A2E (and other bisretinoids) cumulatively called “lipofuscin”, and their accumulation mostly in the RPE. This accumulation of cytotoxic products is a hallmark, and often also the cause, of most phenotypes resulting from dysfunctional ABCA4. More recently, expression of ABCA4 has also been reported in the RPE, suggesting an additional role of the protein in this cell type that, when disturbed, could somehow contribute to ABCA4-associated retinopathy.

There are no approved drugs for Stargardt disease. However, a dual vector gene therapy approach has been proposed. This approach is based on the delivery of two half genes of ABCA4 that combine through trans-splicing or through intein recombination. The trans-splicing/intein technology depends on a continued production of the two recombinant molecules and de novo production of the full length product. An alternative approach is the transplantation of RPE and photoreceptor cells in combination. This option is still many years from clinical trial and is likely to be more suitable for end-stage patients.

In recent years, genome editing to correct mutations in the genome in situ, using systems such as CRISPR/Cas9 (C/c9) or zinc-finger nucleases (ZFN) to create targeted breaks in the genome, has become increasingly popular. However, accurate repair of a mutation to restore the ‘wild-type’ sequence requires that these breaks are repaired through a homologous recombination repair mechanism and this mechanism is not active in neurons such as the photoreceptor cells.

### SUMMARY OF THE INVENTION

The present invention provides a combined genome editing and gene supplementation method that allows the in situ correction of the ABCA4 gene. The advantage of the present invention is that insertion of an ABCA4 partial coding sequence, for example, exon 17-50, into the host chromosome is permanent and will continue to be active during the life of the cell.

The double strand break in ABCA4 created by the editing tool is repaired by the non-homologous end-joining (NHEJ) repair mechanism that predominates in photoreceptors. In a proportion of the cells, the partial coding sequence will be incorporated into the repair reaction, resulting in a hybrid gene that splices from the endogenous sequence to the transgenic/exogenous ABCA4 partial sequence, thus bypassing any mutations present in the downstream endogenous sequence. The tools have been designed such that those repair reactions that have inserted the ABCA4 partial coding sequence are immune from further double strand break formation. In contrast, repairs where the partial coding sequence is not inserted (or inserted in the wrong orientation) may still be cut and repaired again to create a further chance of the partial coding sequence being inserted correctly.

In particular, the invention provides:
[1] A vector system comprising:
(a) a first construct comprising a payload sequence, wherein the payload is a nucleic acid encoding a nuclease; and
(b) a second construct comprising a payload sequence, wherein the payload sequence is a partial human ABCA4 nucleotide sequence.
[2] A pharmaceutical composition comprising the vector system of the invention.
[3] The vector system or the pharmaceutical composition of the invention for use in a method of treating a retinal dystrophy.
[4] A method of treating a retinal dystrophy, the method comprising administering the vector system or the pharmaceutical composition of the invention to a subject,
   optionally wherein the retinal dystrophy is Stargardt disease, cone dystrophy, cone-rod dystrophy, or retinitis pigmentosa,
   further optionally wherein the Stargardt disease is STGD1.
[5] Use of the vector system or the pharmaceutical composition of the invention in the manufacture of a medicament for the treatment of a retinal dystrophy,
   optionally wherein the retinal dystrophy is Stargardt disease, cone dystrophy, cone-rod dystrophy, or retinitis pigmentosa, further optionally wherein the Stargardt disease is STGD1.
[6] A vector comprising a construct encoding a nuclease, for use in simultaneous, separate, or sequential combination with a vector comprising a construct comprising a partial human ABCA4 nucleotide sequence, for the treatment of a retinal dystrophy;
   optionally wherein the retinal dystrophy is Stargardt disease, cone dystrophy, cone-rod dystrophy, or retinitis pigmentosa,
   further optionally wherein the Stargardt disease is STGD1.
[7] A vector comprising a construct comprising a partial human ABCA4 nucleotide sequence, for use in simultaneous, separate, or sequential combination with a vector comprising a construct encoding a nuclease, for the treatment of a retinal dystrophy;
   optionally wherein the retinal dystrophy is Stargardt disease, cone dystrophy, cone-rod dystrophy, or retinitis pigmentosa,
   further optionally wherein the Stargardt disease is STGD1.

### BRIEF DESCRIPTION OF THE FIGURES

FIG. 1: Development of CRISPR/Cas9 that cleaves the human ABCA4 gene

TIDE analysis of ABCA4 CRISPR/Cas9 delivered by AAV-SsH10 to human iPS cell derived retinal organoids. Double strand break formation at the cleavage site was present in 10-20% of genomes for whole EBs, photoreceptors only (CD73+) and remaining cells (CD73−). Note that the average transduction efficiency of AAV-SsH10 in human retinal organoids photoreceptors is ˜20%, suggesting >50% cutting efficiency of genomes in transduced cells.

FIG. 2: Development of CRISPR/Cas9 that cleaves the human ABCA4 gene

Delivery of mouse ABCA4-specific CRISPR/Cas9 in vivo. TIDE analysis shows high efficiency double strand break formation is possible in CD73+ photoreceptors in vivo.

FIG. 3: Insertion of a transgenic ABCA4^{exon17-50} construct into ABCA4 intron 16 in photoreceptor cells in vivo

To show that it is possible to insert a transgenic ABCA4^{exon17-50} construct into ABCA4 intron 16 in photoreceptor cells in vivo, a fusion protein of ABCA4^{exon17-39} with GFP was produced. If inserted in the correct location in the mouse gene, the endogenous ABCA4 expression would drive the partial human ABCA4 sequence and the GFP gene by splicing from the mouse exon 16 to the human exon 17, resulting in GFP protein.

Using AAV carrying the mouse ABCA4 CRISPR construct (AAV.SaCas9) in combination with AAV carrying the donor ABCA4-GFP fusion gene (AAV.donor eGFP), there was GFP protein in a minority of the cells. The same experiment using an AAV-ABCA4-GFP genome flanked by “HITI” CRISPR recognition sites (AAV.donorHITI eGFP) resulted in a substantially greater number of cells expressing GFP, indicating that the inclusion of HITI sites is indeed supporting the correct insertion of the donor genome. The result suggests that it may be feasible to correct enough cells to provide a therapeutic benefit.

FIG. 4: Control experiment using HITI donor construct

Injection of the HITI donor construct in the absence of the AAV-CRISPR/Cas9 vector did not result in cells expressing GFP, indicating that the GFP in the previous figure is not due to leaky expression from the AAV genome, or due to random insertion into the genome.

FIG. 5: ABCA4 staining of retinal organoids

Retinal organoids from Stargardt patient-derived iPS cells (STD) do not stain for the ABCA4 protein, but show otherwise normal morphology.

FIG. 6: In vivo proof of concept of targeted integration

Percentage of GFP+ cells after subretinal injection in WT mice. Two populations of GFP+ cells are identified: Strongly+ cells are absent in retinas transduced with only the ABCA4 donor vector or only the SaCas9 cutting vector. Strongly+ cells increase to 5% of total photoreceptors when using both vectors. Some weakly+ cells are present in retinas transduced with single vectors, but their numbers increase in double transduced retinas. There is a trend for better treatment using Donor vector flanked by CRISPR recognition sites (Donor HITI) relative to Donor without cutting sites (Donor).

FIG. 7: Targeted integration in ABCA4-STD organoids

ABCA4−/− Human retinal organoids (STD) transduced with SaCas9 vector and DonorHITI vector (right) produce greater amounts of ABCA4 protein (white) than organoids transduced with DonorHITI vector only (middle). Wildtype organoids (H9) is provided as a positive control.

FIG. 8: Targeted integration in ABCA4-STD organoids

Independent set of organoids treated identically to those shown in FIG. 7.

FIG. 9: Schematic showing the design of an exemplary vector system

The inventors inserted guide RNA target sequences (including PAM sites) on either side of the ABCA4 partial coding sequence in the therapeutic vector in inverted orientations (see schematic). The SaCas9 will cut the target sequence in intron 16, as well as those in the therapeutic vector. The ABCA4 partial coding sequence is inserted by the cell's DNA repair system into the break in intron 16 in a random orientation. If it is inserted in the correct orientation, the inserted coding sequence will be flanked by two hybrid target sequences (head-head on one side, tail-tail on the other). If the partial coding sequence is inserted in the wrong orientation, it is flanked by two full target sequences (head-tail) which can be cut again by the nuclease for a second attempt at inserting correctly. This can theoretically continue until it inserted in the correct orientation.

FIG. 10: Efficiency of INDEL creation after transfection of 293T cells with a plasmid expressing zinc finger nuclease ZFN16C, targeting intron 16 of the human ABCA4 gene.

TIDE assessment of sequencing traces shows that there is a significantly greater number of INDELs found when comparing zinc finger nuclease treated cells against control cells (ZNF) than when comparing control cells against each other (CTR).

FIG. 11: PCR amplification of ABCA4¹⁷⁻⁵⁰ inserted into intron 16 of the endogenous ABCA4 gene via ZFN16C.
A) Schematic showing design of an experiment to confirm ABCA4¹⁷⁻⁵⁰ insertion into intron 16 of the endogenous ABCA4 gene via ZFN16C.
B) Presence of amplification fragments was assessed by gel electrophoresis. Lane 1: Promega 1 kb ladder; Lane 2: 293T cells transduced with AAV carrying ABCA4¹⁷⁻⁵⁰ without prior transfection with zinc finger nuclease ZFN16C; Lane 3: 293T cells transduced with AAV carrying ABCA4¹⁷⁻⁵⁰ after transfection with zinc finger nuclease ZFN16C. Presence of a 0.48 kb band indicates that there was integration of the recombinant ABCA4¹⁷⁻⁵⁰ into the genomic locus only in the presence of ZFN16C.

### DESCRIPTION OF THE SEQUENCES

-Partial ABCA4 coding sequence.
SEQ ID NO: 1
AATATCTATAGTTACAATTTGAGGTTCAGGTAAATAAACTCTAGATCTTATAGAACTGCGGTAAGGTAGGATA
GGGAGACTCCTTCGACTTTCTCTGTTTATTTGTCTCTATTTTTAGGAGACTATGGAACCCCACTTCCTTGGTA
CTTTCTTCTACAAGAGTCGTATTGGCTTGGCGGTGAAGGGTGTTCAACCAGAGAAGAAAGAGCCCTGGAAAAG
ACCGAGCCCCTAACAGAGGAAACGGAGGATCCAGAGCACCCAGAAGGAATACACGACTCCTTCTTTGAACGTG
AGCATCCAGGGTGGGTTCCTGGGGTATGCGTGAAGAATCTGGTAAAGATTTTTGAGCCCTGTGGCCGGCCAGC
TGTGGACCGTCTGAACATCACCTTCTACGAGAACCAGATCACCGCATTCCTGGGCCACAATGGAGCTGGGAAA
ACCACCACCTTGTCCATCCTGACGGGTCTGTTGCCACCAACCTCTGGGACTGTGCTCGTTGGGGGAAGGGACA
TTGAAACCAGCCTGGATGCAGTCCGGCAGAGCCTTGGCATGTGTCCACAGCACAACATCCTGTTCCACCACCT
CACGGTGGCTGAGCACATGCTGTTCTATGCCCAGCTGAAAGGAAAGTCCCAGGAGGAGGCCCAGCTGGAGATG
GAAGCCATGTTGGAGGACACAGGCCTCCACCACAAGCGGAATGAAGAGGCTCAGGACCTATCAGGTGGCATGC
AGAGAAAGCTGTCGGTTGCCATTGCCTTTGTGGGAGATGCCAAGGTGGTGATTCTGGACGAACCCACCTCTGG
GGTGGACCCTTACTCGAGACGCTCAATCTGGGATCTGCTCCTGAAGTATCGCTCAGGCAGAACCATCATCATG
TCCACTCACCACATGGACGAGGCCGACCTCCTTGGGGACCGCATTGCCATCATTGCCCAGGGAAGGCTCTACT
GCTCAGGCACCCCACTCTTCCTGAAGAACTGCTTTGGCACAGGCTTGTACTTAACCTTGGTGCGCAAGATGAA
AAACATCCAGAGCCAAAGGAAAGGCAGTGAGGGGACCTGCAGCTGCTCGTCTAAGGGTTTCTCCACCACGTGT
CCAGCCCACGTCGATGACCTAACTCCAGAACAAGTCCTGGATGGGGATGTAAATGAGCTGATGGATGTAGTTC
TCCACCATGTTCCAGAGGCAAAGCTGGTGGAGTGCATTGGTCAAGAACTTATCTTCCTTCTTCCAAATAAGAA
CTTCAAGCACAGAGCATATGCCAGCCTTTTCAGAGAGCTGGAGGAGACGCTGGCTGACCTTGGTCTCAGCAGT
TTTGGAATTTCTGACACTCCCCTGGAAGAGATTTTTCTGAAGGTCACGGAGGATTCTGATTCAGGACCTCTGT
TTGCGGGTGGCGCTCAGCAGAAAAGAGAAAACGTCAACCCCCGACACCCCTGCTTGGGTCCCAGAGAGAAGGC
TGGACAGACACCCCAGGACTCCAATGTCTGCTCCCCAGGGGCGCCGGCTGCTCACCCAGAGGGCCAGCCTCCC
CCAGAGCCAGAGTGCCCAGGCCCGCAGCTCAACACGGGGACACAGCTGGTCCTCCAGCATGTGCAGGCGCTGC
TGGTCAAGAGATTCCAACACACCATCCGCAGCCACAAGGACTTCCTGGCGCAGATCGTGCTCCCGGCTACCTT
TGTGTTTTTGGCTCTGATGCTTTCTATTGTTATCCCTCCTTTTGGCGAATACCCCGCTTTGACCCTTCACCCC
TGGATATATGGGCAGCAGTACACCTTCTTCAGCATGGATGAACCAGGCAGTGAGCAGTTCACGGTACTTGCAG
ACGTCCTCCTGAATAAGCCAGGCTTTGGCAACCGCTGCCTGAAGGAAGGGTGGCTTCCGGAGTACCCCTGTGG
CAACTCAACACCCTGGAAGACTCCTTCTGTGTCCCCAAACATCACCCAGCTGTTCCAGAAGCAGAAATGGACA
CAGGTCAACCCTTCACCATCCTGCAGGTGCAGCACCAGGGAGAAGCTCACCATGCTGCCAGAGTGCCCCGAGG
GTGCCGGGGGCCTCCCGCCCCCCCAGAGAACACAGCGCAGCACGGAAATTCTACAAGACCTGACGGACAGGAA
CATCTCCGACTTCTTGGTAAAAACGTATCCTGCTCTTATAAGAAGCAGCTTAAAGAGCAAATTCTGGGTCAAT
GAACAGAGGTATGGAGGAATTTCCATTGGAGGAAAGCTCCCAGTCGTCCCCATCACGGGGGAAGCACTTGTTG
GGTTTTTAAGCGACCTTGGCCGGATCATGAATGTGAGCGGGGGCCCTATCACTAGAGAGGCCTCTAAAGAAAT
ACCTGATTTCCTTAAACATCTAGAAACTGAAGACAACATTAAGGTGTGGTTTAATAACAAAGGCTGGCATGCC
CTGGTCAGCTTTCTCAATGTGGCCCACAACGCCATCTTACGGGCCAGCCTGCCTAAGGACAGGAGCCCCGAGG
AGTATGGAATCACCGTCATTAGCCAACCCCTGAACCTGACCAAGGAGCAGCTCTCAGAGATTACAGTGCTGAC
CACTTCAGTGGATGCTGTGGTTGCCATCTGCGTGATTTTCTCCATGTCCTTCGTCCCAGCCAGCTTTGTCCTT
TATTTGATCCAGGAGCGGGTGAACAAATCCAAGCACCTCCAGTTTATCAGTGGAGTGAGCCCCACCACCTACT
GGGTGACCAACTTCCTCTGGGACATCATGAATTATTCCGTGAGTGCTGGGCTGGTGGTGGGCATCTTCATCGG
GTTTCAGAAGAAAGCCTACACTTCTCCAGAAAACCTTCCTGCCCTTGTGGCACTGCTCCTGCTGTATGGATGG
GCGGTCATTCCCATGATGTACCCAGCATCCTTCCTGTTTGATGTCCCCAGCACAGCCTATGTGGCTTTATCTT
GTGCTAATCTGTTCATCGGCATCAACAGCAGTGCTATTACCTTCATCTTGGAATTATTTGAGAATAACCGGAC
GCTGCTCAGGTTCAACGCCGTGCTGAGGAAGCTGCTCATTGTCTTCCCCCACTTCTGCCTGGGCCGGGGCCTC
ATTGACCTTGCACTGAGCCAGGCTGTGACAGATGTCTATGCCCGGTTTGGTGAGGAGCACTCTGCAAATCCGT
TCCACTGGGACCTGATTGGGAAGAACCTGTTTGCCATGGTGGTGGAAGGGGTGGTGTACTTCCTCCTGACCCT
GCTGGTCCAGCGCCACTTCTTCCTCTCCCAATGGATTGCCGAGCCCACTAAGGAGCCCATTGTTGATGAAGAT
GATGATGTGGCTGAAGAAAGACAAAGAATTATTACTGGTGGAAATAAAACTGACATCTTAAGGCTACATGAAC
TAACCAAGATTTATCCAGGCACCTCCAGCCCAGCAGTGGACAGGCTGTGTGTCGGAGTTCGCCCTGGAGAGTG
CTTTGGCCTCCTGGGAGTGAATGGTGCCGGCAAAACAACCACATTCAAGATGCTCACTGGGGACACCACAGTG
ACCTCAGGGGATGCCACCGTAGCAGGCAAGAGTATTTTAACCAATATTTCTGAAGTCCATCAAAATATGGGCT
ACTGTCCTCAGTTTGATGCAATTGATGAGCTGCTCACAGGACGAGAACATCTTTACCTTTATGCCCGGCTTCG
AGGTGTACCAGCAGAAGAAATCGAAAAGGTTGCAAACTGGAGTATTAAGAGCCTGGGCCTGACTGTCTACGCC
GACTGCCTGGCTGGCACGTACAGTGGGGGCAACAAGCGGAAACTCTCCACAGCCATCGCACTCATTGGCTGCC
CACCGCTGGTGCTGCTGGATGAGCCCACCACAGGGATGGACCCCCAGGCACGCCGCATGCTGTGGAACGTCAT
CGTGAGCATCATCAGAGAAGGGAGGGCTGTGGTCCTCACATCCCACAGCATGGAAGAATGTGAGGCACTGTGT
ACCCGGCTGGCCATCATGGTAAAGGGCGCCTTTCGATGTATGGGCACCATTCAGCATCTCAAGTCCAAATTTG
GAGATGGCTATATCGTCACAATGAAGATCAAATCCCCGAAGGACGACCTGCTTCCTGACCTGAACCCTGTGGA
GCAGTTCTTCCAGGGGAACTTCCCAGGCAGTGTGCAGAGGGAGAGGCACTACAACATGCTCCAGTTCCAGGTC
TCCTCCTCCTCCCTGGCGAGGATCTTCCAGCTCCTCCTCTCCCACAAGGACAGCCTGCTCATCGAGGAGTACT
CAGTCACACAGACCACACTGGACCAGGTGTTTGTAAATTTTGCTAAACAGCAGACTGAAAGTCATGACCTCCC
TCTGCACCCTCGAGCTGCTGGAGCCAGTCGACAAGCCCAGGACTGA

In italics: 3′ 118 bp of Intron 16

Alternating bold and underlined: Exons 17 to 50

-gRNAHu1 target sequence
SEQ ID NO: 2
TAAAGATCCAGACCTGCCCCGAGGAAT
-gRNAHu2 target sequence
SEQ ID NO: 3
CTTATAAGGATACCAACTGGATTGGAT

The PAM site is underlined and the region which is complementary to the guide RNA is in bold.

-SaCas9
SEQ ID NO: 4
AAGCGGAACTACATCCTGGGCCTGGACATCGGCATCACCAGCGTGGGCTACGGCATCATCGACTACGAGACAC
GGGACGTGATCGATGCCGGCGTGCGGCTGTTCAAAGAGGCCAACGTGGAAAACAACGAGGGCAGGCGGAGCAA
GAGAGGCGCCAGAAGGCTGAAGCGGCGGAGGCGGCATAGAATCCAGAGAGTGAAGAAGCTGCTGTTCGACTAC
AACCTGCTGACCGACCACAGCGAGCTGAGCGGCATCAACCCCTACGAGGCCAGAGTGAAGGGCCTGAGCCAGA
AGCTGAGCGAGGAAGAGTTCTCTGCCGCCCTGCTGCACCTGGCCAAGAGAAGAGGCGTGCACAACGTGAACGA
GGTGGAAGAGGACACCGGCAACGAGCTGTCCACCAAAGAGCAGATCAGCCGGAACAGCAAGGCCCTGGAAGAG
AAATACGTGGCCGAACTGCAGCTGGAACGGCTGAAGAAAGACGGCGAAGTGCGGGGCAGCATCAACAGATTCA
AGACCAGCGACTACGTGAAAGAAGCCAAACAGCTGCTGAAGGTGCAGAAGGCCTACCACCAGCTGGACCAGAG
CTTCATCGACACCTACATCGACCTGCTGGAAACCCGGCGGACCTACTATGAGGGACCTGGCGAGGGCAGCCCC
TTCGGCTGGAAGGACATCAAAGAATGGTACGAGATGCTGATGGGCCACTGCACCTACTTCCCCGAGGAACTGC
GGAGCGTGAAGTACGCCTACAACGCCGACCTGTACAACGCCCTGAACGACCTGAACAATCTCGTGATCACCAG
GGACGAGAACGAGAAGCTGGAATATTACGAGAAGTTCCAGATCATCGAGAACGTGTTCAAGCAGAAGAAGAAG
CCCACCCTGAAGCAGATCGCCAAAGAAATCCTCGTGAACGAAGAGGATATTAAGGGCTACAGAGTGACCAGCA
CCGGCAAGCCCGAGTTCACCAACCTGAAGGTGTACCACGACATCAAGGACATTACCGCCCGGAAAGAGATTAT
TGAGAACGCCGAGCTGCTGGATCAGATTGCCAAGATCCTGACCATCTACCAGAGCAGCGAGGACATCCAGGAA
GAACTGACCAATCTGAACTCCGAGCTGACCCAGGAAGAGATCGAGCAGATCTCTAATCTGAAGGGCTATACCG
GCACCCACAACCTGAGCCTGAAGGCCATCAACCTGATCCTGGACGAGCTGTGGCACACCAACGACAACCAGAT
CGCTATCTTCAACCGGCTGAAGCTGGTGCCCAAGAAGGTGGACCTGTCCCAGCAGAAAGAGATCCCCACCACC
CTGGTGGACGACTTCATCCTGAGCCCCGTCGTGAAGAGAAGCTTCATCCAGAGCATCAAAGTGATCAACGCCA
TCATCAAGAAGTACGGCCTGCCCAACGACATCATTATCGAGCTGGCCCGCGAGAAGAACTCCAAGGACGCCCA
GAAAATGATCAACGAGATGCAGAAGCGGAACCGGCAGACCAACGAGCGGATCGAGGAAATCATCCGGACCACC
GGCAAAGAGAACGCCAAGTACCTGATCGAGAAGATCAAGCTGCACGACATGCAGGAAGGCAAGTGCCTGTACA
GCCTGGAAGCCATCCCTCTGGAAGATCTGCTGAACAACCCCTTCAACTATGAGGTGGACCACATCATCCCCAG
AAGCGTGTCCTTCGACAACAGCTTCAACAACAAGGTGCTCGTGAAGCAGGAAGAAAACAGCAAGAAGGGCAAC
CGGACCCCATTCCAGTACCTGAGCAGCAGCGACAGCAAGATCAGCTACGAAACCTTCAAGAAGCACATCCTGA
ATCTGGCCAAGGGCAAGGGCAGAATCAGCAAGACCAAGAAAGAGTATCTGCTGGAAGAACGGGACATCAACAG
GTTCTCCGTGCAGAAAGACTTCATCAACCGGAACCTGGTGGATACCAGATACGCCACCAGAGGCCTGATGAAC
CTGCTGCGGAGCTACTTCAGAGTGAACAACCTGGACGTGAAAGTGAAGTCCATCAATGGCGGCTTCACCAGCT
TTCTGCGGCGGAAGTGGAAGTTTAAGAAAGAGCGGAACAAGGGGTACAAGCACCACGCCGAGGACGCCCTGAT
CATTGCCAACGCCGATTTCATCTTCAAAGAGTGGAAGAAACTGGACAAGGCCAAAAAAGTGATGGAAAACCAG
ATGTTCGAGGAAAAGCAGGCCGAGAGCATGCCCGAGATCGAAACCGAGCAGGAGTACAAAGAGATCTTCATCA
CCCCCCACCAGATCAAGCACATTAAGGACTTCAAGGACTACAAGTACAGCCACCGGGTGGACAAGAAGCCTAA
TAGAGAGCTGATTAACGACACCCTGTACTCCACCCGGAAGGACGACAAGGGCAACACCCTGATCGTGAACAAT
CTGAACGGCCTGTACGACAAGGACAATGACAAGCTGAAAAAGCTGATCAACAAGAGCCCCGAAAAGCTGCTGA
TGTACCACCACGACCCCCAGACCTACCAGAAACTGAAGCTGATTATGGAACAGTACGGCGACGAGAAGAATCC
CCTGTACAAGTACTACGAGGAAACCGGGAACTACCTGACCAAGTACTCCAAAAAGGACAACGGCCCCGTGATC
AAGAAGATTAAGTATTACGGCAACAAACTGAACGCCCATCTGGACATCACCGACGACTACCCCAACAGCAGAA
ACAAGGTCGTGAAGCTGTCCCTGAAGCCCTACAGATTCGACGTGTACCTGGACAATGGCGTGTACAAGTTCGT
GACCGTGAAGAATCTGGATGTGATCAAAAAAGAAAACTACTACGAAGTGAATAGCAAGTGCTATGAGGAAGCT
AAGAAGCTGAAGAAGATCAGCAACCAGGCCGAGTTTATCGCCTCCTTCTACAACAACGATCTGATCAAGATCA
ACGGCGAGCTGTATAGAGTGATCGGCGTGAACAACGACCTGCTGAACCGGATCGAAGTGAACATGATCGACAT
CACCTACCGCGAGTACCTGGAAAACATGAACGACAAGAGGCCCCCCAGGATCATTAAGACAATCGCCTCCAAG
ACCCAGAGCATTAAGAAGTACAGCACAGACATTCTGGGCAACCTGTATGAAGTGAAATCTAAGAAGCACCCTC
AGATCATCAAAAAGGGC
-reverse complement of gRNAHul target sequence
SEQ ID NO: 5
ATTCCTCGGGGCAGGTCTGGATCTTTA
-reverse complement of gRNAHu2 target sequence
SEQ ID NO: 6
ATCCAATCCAGTTGGTATCCTTATAAG
-Amino acid sequence of ZFN16C
SEQ ID NO: 7
MPAAKRVKLDYACPVESCDRRFSTSGHLVRHIRIHTGEKPFQCRICMRNFSRDSHLSRHIRTHTGEKPFACDI
CGRKFATSANLSRHTKIHTGQKDQLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAREMQRYVEENQTRNKHINPNEWWK
VYPSSVTEFKFLFVSGHFKGNYKAQLTRLNHITNCNGAVLSVEELLIGGEMIKAGTLTLEEVRRKFNNGEINF
GSGATNFSLLKQAGDVEENPGPPAAKRVKLDYACPVESCDRRFSTSANLSRHIRIHTGEKPFQCRICMRNFSR
NDALTEHIRTHTGEKPFACDICGRKFAQNSTLTEHTKIHTGQKDQLVKSELEEKKSELRHKLKYVPHEYIELI
EIARNSTQDRILEMKVMEFFMKVYGYRGKHLGGSRKPDGAIYTVGSPIDYGVIVDTKAYSGGYNLPIGQAREM
QRYVEENQTRNKHINPNEWWKVYPSSVTEFKFLFVSGHFKGNYKAQLTRLNHITNCNGAVLSVEELLIGGEMI
KAGTLTLEEVRRKFNNGEINF
-Nucleotide sequence of ZFN16C
SEQ ID NO: 8
ATGCCCGCCGCCAAGCGGGTGAAGCTGGACTACGCCTGCCCAGTGGAAAGTTGTGACCGG
CGGTTCAGCCGCGACAGCCACCTGTCCCGCCACATCCGGATCCATACAGGCGAGAAACCT
TTCCAGTGCAGAATCTGCATGAGAAATTTCAGCCAGAGCAGCTCTCTGGTGCGGCACATC
AGAACCCACACAGGAGAGAAGCCTTTCGCCTGCGATATCTGTGGAAGAAAGTTCGCCACC
TCCGGACACCTTGTGAGACATACAAAAATCCACACAGGCTCTGAGAGACCTTATGCCTGC
CCTGTGGAGTCTTGTGACAGACGGTTCAGCAGAGATAGCCACCTGAGCAGACATATCAGA
ATCCATACAGGCGAGAAGCCCTTTCAGTGCCGGATCTGCATGAGAAACTTCAGTACAAGC
GCCAATCTGAGCAGACACATCCGGACCCACACCGGACAGAAAGACCAGCTGGTGAAGTCT
GAGCTGGAAGAAAAGAAGAGCGAACTGAGACACAAGCTGAAGTACGTGCCACACGAGTAC
ATCGAGCTGATCGAAATCGCCCGGAACAGCACCCAGGATCGGATTCTGGAAATGAAGGTG
ATGGAATTCTTCATGAAAGTGTATGGCTACAGAGGCAAACACCTGGGCGGCAGCAGAAAA
CCTGATGGCGCCATCTACACCGTTGGATCTCCTATCGACTACGGCGTGATTGTCGACACA
AAGGCCTACAGCGGCGGGTACAACCTGCCTATCGGCCAGGCCAGAGAGATGCAGCGGTAC
GTGGAGGAAAACCAGACCAGAAACAAGCACATCAACCCCAACGAGTGGTGGAAGGTGTAT
CCTAGCTCCGTGACCGAGTTCAAGTTCCTGTTCGTGTCCGGCCACTTCAAGGGCAACTAC
AAGGCTCAGCTAACCCGCCTCAACCACATCACCAATTGCAATGGCGCTGTTCTGTCTGTG
GAAGAGCTGCTGATCGGCGGCGAGATGATTAAGGCCGGCACCCTGACCCTGGAGGAAGTG
AGAAGAAAGTTTAACAACGGCGAAATCAACTTCGGCTCTGGCGCCACCAACTTTTCTCTG
CTGAAACAGGCCGGCGACGTGGAGGAGAACCCCGGCCCTCCTGCCGCTAAACGGGTGAAA
CTGGATTACGCGTGTCCCGTGGAATCCTGCGATAGAAGATTCTCTAGAAGCGACCACCTG
AGCAGACACATCCGGATCCACACCGGCGAAAAGCCCTTCCAGTGCCGGATCTGCATGCGG
AACTTCAGCACCCTGAGCCTGCACACCGAACACATCCGGACCCACACAGGCGAGAAGCCA
TTCGCCTGTGATATCTGTGGCAGGAAGTTCGCCCAGAACAGCACCCTGACCGAGCACACC
AAGATCCACACCGGCAGCGAGCGGCCTTACGCCTGCCCTGTCGAGAGCTGCGATCGGCGA
TTTTCCACCAGCGCCAACCTCAGCAGGCATATCAGAATCCACACAGGCGAGAAACCTTTT
CAGTGTAGAATCTGCATGAGAAACTTCAGCAGGAACGACGCCCTGACAGAGCACATCAGA
ACCCACACCGGAGAAAAGCCGTTCGCCTGCGACATCTGCGGTAGAAAATTCGCTCAAAAT
AGCACACTGACAGAGCACACCAAGATCCACACTGGACAAAAGGACCAGCTGGTCAAGAGC
GAGCTCGAAGAGAAGAAAAGCGAGCTGAGACATAAGCTGAAGTATGTGCCTCACGAGTAC
ATCGAGCTGATCGAGATCGCTAGAAACAGCACCCAGGACAGAATCCTGGAGATGAAGGTG
ATGGAATTTTTCATGAAGGTGTACGGCTACCGGGGCAAGCACCTGGGCGGATCTCGGAAA
CCTGACGGCGCCATCTACACCGTGGGCTCCCCAATTGACTACGGCGTGATCGTGGACACC
AAGGCTTACAGCGGCGGATACAACCTGCCCATCGGCCAGGCTAGAGAGATGCAGAGATAC
GTGGAAGAGAATCAGACAAGAAACAAGCACATCAACCCTAATGAGTGGTGGAAGGTGTAC
CCCAGCAGCGTGACAGAATTCAAGTTCCTGTTCGTGTCTGGCCACTTTAAGGGCAATTAC
AAGGCCCAACTGACCAGACTGAACCACATCACCAACTGCAACGGCGCCGTGCTGAGCGTG
GAAGAGCTGCTGATTGGAGGAGAGATGATTAAGGCCGGCACACTGACCCTGGAAGAAGTG
CGGAGAAAGTTCAACAACGGCGAAATCAACTTCTAA
-Forward amplification primer for ZFN16C
SEQ ID NO 9
GAAAGGAAACAGAGGCACAC
-Reverse amplification primer for ZFN16C
SEQ ID NO: 10
AGATAAAGATCCAGACCTGCC
-Forward amplification primer for ABCA4 template insertion
SEQ ID NO: 11
AGAAAGGAAACAGAGGCACAC
-Reverse amplification primer for ABCA4 template insertion
SEQ ID NO: 12
TTCACGCATACCCCAGGAAC

### DETAILED DESCRIPTION

### Definitions

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and can be isolated from genes, or chemically synthesized by methods known in the art. The terms “polynucleotide” and “nucleic acid” should be understood to include, as applicable to the aspects being described, single-stranded (such as sense or antisense) and double-stranded polynucleotides.

“Genomic DNA” refers to the DNA of a genome of an organism including, but not limited to, the DNA of the genome of a bacterium, fungus, archea, plant or animal.

By “hybridizable” or “complementary” or “substantially complementary” it is meant that a nucleic acid (e.g. RNA) comprises a sequence of nucleotides that enables it to non-covalently bind, e.g.: form Watson-Crick base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation. It is understood in the art that the sequence of polynucleotide need not be 100% complementary for hybridization. Percent complementarity between particular stretches of nucleic acid sequences within nucleic acids can be determined routinely using BLAST programs (basic local alignment search tools) and PowerBLAST programs known in the art (Altschul et ah, J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include naturally occurring amino acids, chemically or biochemically modified or derivatized amino acids, and polypeptides having modified peptide backbones.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST), available over the world wide web at sites including ncbi.nlm.nili.gov/BLAST. See, e.g., Altschul et al. (1990), J. Mol. Bio. 215:403-10.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (e.g., spacer or spacer sequence) of a guide RNA will bind, provided suitable conditions for binding exist. Suitable DNA/RNA binding conditions include physiological conditions normally present in a cell. Other suitable DNA/RNA binding conditions (e.g., conditions in a cell-free system) are known in the art.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair).

The terms “treatment”, “treating” and the like are used herein to generally mean obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which can be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent can be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

It is appreciated that certain features of the invention, which are described in the context of separate examples, can also be provided in combination in a single example. Conversely, various features of the invention, which are, for brevity, described in the context of a single example, can also be provided separately or in any suitable sub-combination. All combinations of the examples pertaining to the disclosure are specifically embraced.

Throughout this specification, the word “comprise”, or variations such as “comprised” or “comprising”, will be understood to imply the inclusion of a stated element, integer or step, or group of elements, integers or steps, but not the exclusion of any other element, integer or step, or group of elements, integers or steps.

In addition as used in this specification and the appended claims, the singular forms “a”, “an”, and “the” include plural references unless the content clearly dictates otherwise.

Thus, for example, reference to “vector” includes “vectors”, and the like.

All publications, patents and patent applications cited herein, whether supra or infra, are hereby incorporated by reference in their entirety.

### Vector System

The invention provides a vector system comprising:
(a) a first construct comprising a payload sequence, wherein the payload sequence is a nucleic acid encoding a nuclease; and
(b) a second construct comprising a payload sequence, wherein the payload sequence is a partial human ABCA4 nucleotide sequence.

### First Construct—Genome Editing Tool

The invention provides a first construct comprising a payload sequence, wherein the payload sequence is a nucleic acid encoding a nuclease and/or a genome editing tool. The nuclease of the invention may be any nuclease suitable for genome editing. For example, the nuclease may be selected from a CRISPR nuclease, a transcription activator-like effector nuclease (TALEN), a DNA-guided nuclease, a meganuclease, or a Zinc Finger Nuclease (ZFN).

A ZFN is a heterodimer in which each subunit contains a zinc finger domain and a FokI endonuclease domain. ZFNs constitute the largest individual family of transcriptional modulators known for higher organisms. In certain embodiments, the payload sequence comprises a DNA-binding domain made up of Cys2His2 zinc fingers fused to a KRAB repressor. In a preferred embodiment of the invention, the payload sequence comprises a zinc-finger-KRAB sequence. ZFNs do not use a guide RNA. Nevertheless, ZFNs can be created specific for any cutting site. This feature, in addition to their small size, makes ZFNs a preferred nuclease of the invention.

In a preferred embodiment of the invention, the first construct comprises a payload sequence, wherein the payload sequence is a nucleic acid encoding a ZFN. In a preferred embodiment of the invention the first construct comprises a payload sequence, wherein the payload sequence is a nucleic acid encoding a ZFN targeting an intron of ABCA4. In a preferred embodiment of the invention the first construct comprises a payload sequence, wherein the payload sequence is a nucleic acid encoding a ZFN targeting intron 16 of ABCA4.

In a preferred embodiment of the invention, the first construct comprises a payload sequence, wherein the payload sequence is a nucleic acid which encodes a polypeptide having at least 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity or 99% sequence identity to SEQ ID NO: 7, said polypeptide variants maintaining the ability to function as ZFNs.

In a preferred embodiment, the first construct comprises a payload sequence, wherein the payload sequence is a nucleic acid which encodes SEQ ID NO:7.

In a preferred embodiment of the invention, the first construct comprises a payload sequence, wherein the payload sequence is a nucleic acid having at least 60% sequence identity, 65% sequence identity, 70% sequence identity, 75% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity or 99% sequence identity to SEQ ID NO: 8, said nucleic acid variants maintaining the ability to encode functional ZFNs.

In a preferred embodiment of the invention, the first construct comprises a payload sequence, wherein the payload sequence is a nucleic acid sequence comprising SEQ ID NO: 8. In a preferred embodiment of the invention, the first construct comprises a payload sequence, wherein the payload sequence is a nucleic acid sequence consisting of SEQ ID NO: 8.

TALENs comprise a non-specific DNA-cleaving nuclease fused to a DNA-binding domain that can be customised so that TALENs can target a sequence of interest to be silenced (Joung and Sander, 2013). In certain embodiments, the payload sequence comprises a TALEN sequence. TALENs do not use a guide RNA.

Naturally-occurring CRISPR/Cas systems are genetic defence systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et ah, (2017) Curr Opin Microbiol 37:67-78).

Engineered versions of CRISPR/Cas systems has been developed in numerous formats to mutate or edit genomic DNA of cells from other species. CRISPR/Cas systems comprise at least two components: 1) a Cas nuclease and 2) a guide RNA (gRNA). The general approach of using the CRISPR/Cas system involves the heterologous expression or introduction of a site-directed nuclease (e.g.: Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g. genes).

In some embodiments, the Cas nuclease is Cas9 or a Cas9 ortholog. Exemplary species that the Cas9 nuclease may be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium dijficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina.

In some embodiments, the Cas9 protein is from Streptococcus pyogenes (SpCas9), Streptococcus thermophilus (StCas9), Neisseria meningitides (NmCas9), Staphylococcus aureus (SaCas9), or Campylobacter jejuni (CjCas9). Of these nucleases, SaCas9 is particularly preferred due to the relatively smaller size which allows the guide RNA and nuclease to fit in a single vector, such as an AAV vector. Furthermore, a SaCas9 target site can be found in intron 16 of ABCA4.

In a preferred embodiment of the invention, the first construct comprises a payload sequence, wherein the payload sequence is a nucleic acid encoding an SaCas9. In a preferred embodiment of the invention, the first construct comprises a payload sequence, wherein the payload sequence is a nucleic acid having at least 60% sequence identity, 65% sequence identity, 70% sequence identity, 75% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity or 99% sequence identity to SEQ ID NO: 4, said nucleic acid variants maintaining the ability to encode a functional SaCas9.

In a preferred embodiment of the invention, the first construct comprises a payload sequence, wherein the payload sequence is a nucleic acid sequence comprising SEQ ID NO: 4. In a preferred embodiment of the invention, the first construct comprises a payload sequence, wherein the payload sequence is a nucleic acid sequence consisting of SEQ ID NO: 4.

In some embodiments, Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fokl. A Cas9 nuclease is a modified nuclease.

In some embodiments, the nucleic acid encoding the nuclease is codon optimized for efficient expression in one or more eukaryotic cell types. In some embodiments, the nucleic acid encoding the nuclease is codon optimized for efficient expression in one or more mammalian cells. In some embodiments, the nucleic acid encoding the nuclease is codon optimized for efficient expression in human cells. Methods of codon optimization including codon usage tables and codon optimization algorithms are available in the art.

The second component of the CRISPR/Cas system is the guide RNA (gRNA). The gRNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence by virtue of its association with the protein-binding segment of the gRNA. In engineered CRISPR/Cas systems, a gRNA/Cas nuclease complex is targeted to a specific target sequence of interest within a target nucleic acid (e.g. a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA:tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA provided by the disclosure comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule gRNA. Alternatively, the crRNA and tracr RNA may be transcribed into a single-molecule gRNA. In other embodiments, the crRNA and the tracr RNA may be driven by their corresponding promoters on the same vector. In yet other embodiments, the crRNA and the tracr RNA may be encoded by different vectors.

In some embodiments, the nucleotide sequence encoding the gRNA may be located on the same vector comprising the nucleotide sequence encoding a nuclease. In some embodiments, expression of the gRNA and of the nuclease may be driven by different promoters. In some embodiments, expression of the gRNA may be driven by the same promoter that drives expression of the nuclease. In some embodiments, the gRNA and the nuclease transcript may be contained within a single transcript. For example, the guide RNA may be within an untranslated region (UTR) of the nuclease transcript. In some embodiments, the gRNA may be within the 5′ UTR of the nuclease protein transcript. In other embodiments, the gRNA may be within the 3′ UTR of the nuclease transcript. In some embodiments of the invention one or more gRNAs are used. In some embodiments of the invention multiple gRNAs are used. In some embodiments of the invention two or more gRNAs are used.

In a preferred embodiment of the invention, a single gRNA is used. The gRNA is designed to create a double strand break (DSB) the ABCA4 gene. The partial ABCA4 coding sequence is inserted into the DSB. In this embodiment, the 3′ portion of the endogenous ABCA4 gene is not removed with a second cut, but is left in place as junk. Suitable gRNAs may be designed by the skilled person using design tools, such as Benchling.com. Candidate gRNA sequences targeting ABCA4 may be chosen based on cutting efficiency predicted by the design tool algorithm.

In some embodiments, the gRNA is between 10-30, or between 15-25, or between 15-20 nucleotides in length.

The complementary strand of the target sequence is complementary to spacer sequence of the gRNA. In some embodiments, the degree of complementarity between the spacer sequence of a gRNA and its corresponding complementary strand of the target sequence is about 50%, 55%, 60%, 65%, 70%, 75%, 80%, 85%, 90%, 95%, 97%, 98%, 99%, or 100%. In some embodiments, the complementary strand of the target sequence and the spacer sequence is 100% complementary. In other embodiments, the complementary strand of the target sequence and the spacer sequence of the gRNA contains at least one mismatch. For example, the complementary strand of the target sequence and the spacer sequence of the guide RNA contain 1, 2, 3, 4, 5, 6, 7, 8, 9, or 10 mismatches. In some embodiments, the complementary strand of the target sequence and the spacer sequence of the guide RNA contain 1-6 mismatches. In some embodiments, the complementary strand of the target sequence and the targeting sequence of the guide RNA contain 5 or 6 mismatches.

In some embodiments, the target sequence may be adjacent to a protospacer adjacent motif (PAM), a short sequence recognized by a CRISPR/Cas9 complex. In some embodiments, the PAM may be adjacent to or within 1, 2, 3, or 4, nucleotides of the 3′ end of the target sequence. The length and the sequence of the PAM may depend on the Cas9 protein used. For example, the PAM may be selected from a consensus or a particular PAM sequence for a specific Cas9 nuclease or Cas9 ortholog. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fokl, SpCas9-HFI, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT orNNGRRN (SaCas9), NNNRRT (KKH SaCas9), NNNNRYAC (CjCas9), NNAGAAW (StlCas9), NAAAAC (TdCas9), NGGNG (St3Cas9), NG (FnCas9), N AAA AN (TdCas9), NNAAAAW (StCas9), NNNNACA (CjCas9), GNNNCNNA (PmCas9), and NNNNGATT (NmCas9) (wherein N is defined as any nucleotide, W is defined as either A or T, R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

In one embodiment of the invention, the gRNA designed to target a double strand break (DSB) to the ABCA4 gene is SEQ ID NO: 2. In one embodiment of the invention, the gRNA designed to target a double strand break (DSB) to the ABCA4 gene is SEQ ID NO: 3.

In one embodiment of the invention, an expression cassette producing gRNA that is complementary to SEQ ID NO: 2 is present in the same vector as the nuclease. In one embodiment of the invention, an expression cassette producing gRNA that is complementary to SEQ ID NO: 3 is present in the same vector as the nuclease.

In one embodiment of the invention, an expression cassette producing gRNA that is complementary to SEQ ID NO: 2 is present in the same vector as the nucleic acid sequence comprising SEQ ID NO: 4. In one embodiment of the invention, an expression cassette producing gRNA that is complementary to SEQ ID NO: 3 is present in the same vector as the nucleic acid sequence comprising SEQ ID NO: 4.

In one embodiment of the invention, the first construct encodes a CRISPR nuclease and additionally comprises a nucleic acid sequence encoding a guide RNA (gRNA) comprising a sequence that is complementary to a target sequence within intron 16 of the endogenous human ABCA4 gene.

In one embodiment of the invention, the first construct encodes a CRISPR nuclease and additionally comprises a nucleic acid sequence encoding a guide RNA (gRNA) containing a sequence complementary to SEQ ID NO: 2.

In one embodiment of the invention, the first construct encodes a CRISPR nuclease and additionally comprises a nucleic acid sequence encoding a guide RNA (gRNA) containing a sequence complementary to SEQ ID NO: 3.

In one embodiment of the invention, the polynucleotide encoding the nuclease is operably linked to a promoter. The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art. Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the construct can depend on such factors as the choice of the target cell, the level of expression desired, and the like. In some embodiments, the promoter must also be small enough to fit into the vector together with the gRNA and the cDNA encoding the nuclease.

Vectors used for providing the nucleic acids encoding gRNA and nuclease to the cell typically comprises a suitable promoter for driving the expression of the nucleic acid of interest. In other words, the nucleic acid of interest will be operably linked to a promoter. This can include ubiquitously acting promoters, inducible promoters, or promoters that are preferably or specifically active in particular cell populations, such as photoreceptor cells. For example, the promoter may be a photoreceptor-specific or photoreceptor-preferred promoter, more preferably a rod-specific or rod-preferred promoter such as a Rhodopsin (Rho), Neural retina-specific leucine zipper protein (NRL) or Phosphodiesterase 6B (PDE6B) promoter.

By a photoreceptor-specific promoter, is meant a promoter that drives expression only or substantially only in photoreceptors, e.g. one that drives expression at least a hundred-fold more strongly in photoreceptors than in any other cell type. By a rod-specific promoter, is meant a promoter that drives expression only or substantially only in photoreceptors, e.g. one that drives expression at least a hundred-fold more strongly in photoreceptors than in any other cell type, including cones. By a photoreceptor-preferred promoter, is meant a promoter that expresses preferentially in photoreceptors but may also drive expression to some extent in other tissues, e.g. one that drives expression at least two-fold, at least five-fold, at least ten-fold, at least 20-fold, or at least 50-fold more strongly in photoreceptors than in any other cell type. By a rod-preferred promoter, is meant a promoter that drives expression preferentially in photoreceptors but may also drive expression to some extent in other tissues, e.g. one that drives expression at least two-fold, at least five-fold, at least ten-fold, at least 20-fold, or at least 50-fold more strongly in photoreceptors than in any other cell type. including cones.

The promoter region incorporated into the construct may be of any length as long as it is effective to drive expression of the gene product, preferably photoreceptor-specific or photoreceptor-preferred expression or rod-specific or rod-preferred expression.

In some embodiments, the nucleotide sequence encoding the gRNA may be operably linked to at least one transcriptional or translational control sequence. In some embodiments, the nucleotide sequence encoding the gRNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the gRNA may be operably linked to a mouse or human U6 promoter. In other embodiments, the nucleotide sequence encoding the gRNA may be operably linked to a mouse or human HI promoter. In some embodiments, the nucleotide sequence encoding the guide RNA may be operably linked to a mouse or human tRNA promoter. In embodiments with more than one gRNA, the promoters used to drive expression may be the same or different.

### Second Construct—ABCA4 Sequence

The invention provides a second construct comprising a payload sequence, wherein the payload sequence is a partial human ABCA4 nucleotide sequence. In particular, the ABCA4 sequence is a DNA sequence, such as a genomic or cDNA sequence. cDNA sequences are preferred due to the size limitations imposed by the vector.

The ABCA4 sequence used in the system of the invention preferably corresponds to the ABCA4 cDNA sequence downstream from the chosen intronic target site. For example, the partial ABCA4 sequence may comprise exons 35-50, exons 30-50, exons 25-50, exons 20-50, or exons 17-50 of ABCA4. For example, the partial ABCA4 sequence may comprise exons 17-50, exons 18-50, exons 19-50, exons 20-50, exons 21-50, exons 21-50, exons 23-50, exons 24-50, exons 25-50, exons 26-50, exons 27-50, exons 28-50, exons 29-50, exons 30-50, exons 31-50, exons 32-50, exons 33-50, exons 34-50, exons 35-50, exons 36-50, exons 37-50, exons 38-50, exons 39-50, exons 40-50, exons 41-50, exons 42-50, exons 43-50, exons 44-50, exons 45-50, exons 46-50, exons 47-50, exons 48-50, exons 49-50 or exon 50 of the ABCA4 sequence.

The ABCA4 polynucleotide sequence comprises a partial wildtype ABCA4 sequence, or a sequence having at least 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity or 99% sequence identity to the corresponding partial wildtype ABCA4 sequence, wherein integration of the partial sequence into the genome of a subject restores the expression and/or function of ABCA4.

In a preferred embodiment, the partial sequence comprises exons 17-50 of ABCA4. In a preferred embodiment, the partial sequence comprises a sequence having at least 60% sequence identity, 65% sequence identity, 70% sequence identity, 75% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity or 99% sequence identity to SEQ ID NO: 1, wherein integration of the partial sequence into the genome of a subject restores the expression and/or function of ABCA4. In a preferred embodiment, the partial sequence comprises SEQ ID NO:1. In a preferred embodiment, the partial sequence consists of SEQ ID NO:1.

Described herein, the polynucleotide encoding the ABCA4 partial sequence may be modified by inserting guide RNA target sequences, including the PAM sites, on either side of the coding sequence in inverted orientations (see FIG. 9). The nuclease will cut the target sequences in the genome and in the vector comprising the partial ABCA4 coding sequence. The ABCA4 partial coding sequence is then inserted by the cell's DNA repair system into the DSB in a random orientation by NHEJ. If it is inserted in the correct orientation, the inserted coding sequence will be flanked by two hybrid target sequences (head-head on one side, tail-tail on the other). If the partial coding sequence is inserted in the wrong orientation, it is flanked by two full target sequences (head-tail) which can be cut again by the nuclease for a second attempt at inserting correctly. This can theoretically continue until it inserted in the correct orientation. The method described in the paragraph above can be considered to be a homology-independent targeted integration (HITI) method.

In a preferred embodiment, the guide RNA target sequences inserted on either side of the polynucleotide encoding the ABCA4 partial sequence comprise or consist of SEQ ID NO:5. In a preferred embodiment, the guide RNA target sequences inserted on either side of the polynucleotide encoding the ABCA4 partial sequence comprise or consist of SEQ ID NO:6.

In a preferred embodiment of the invention, the second construct comprises in a 5′ to 3′ direction:
a) SEQ ID NO: 5;
b) SEQ ID NO: 1, or a sequence having at least 80% sequence identity thereto; and
c) SEQ ID NO: 5.

In a preferred embodiment of the invention, the second construct comprises in a 5′ to 3′ direction:
a) SEQ ID NO: 5;
b) SEQ ID NO: 1, or a sequence having at least 90% sequence identity thereto; and
c) SEQ ID NO: 5.

In a preferred embodiment of the invention, the second construct comprises in a 5′ to 3′ direction:
a) SEQ ID NO: 6;
b) SEQ ID NO: 1, or a sequence having at least 80% sequence identity thereto; and
c) SEQ ID NO: 6.

In a preferred embodiment of the invention, the second construct comprises in a 5′ to 3′ direction:
a) SEQ ID NO: 6;
b) SEQ ID NO: 1, or a sequence having at least 90% sequence identity thereto; and
c) SEQ ID NO: 6.

In contrast to the construct or vector encoding the nuclease and/or gRNA, the construct or vector comprising the partial ABCA4 polynucleotide sequence does not comprise a promoter sequence. Instead, the expression of the ABCA4 polynucleotide sequence is driven by the endogenous ABCA4 promoter once inserted into the target site in the genome.

### Vector

In some embodiments, the nucleotide sequences encoding a nuclease and the ABCA4 polynucleotide may be located on the same or separate vectors. In some embodiments, the vector encoding the nuclease additionally encodes a gRNA. In a preferred embodiment, the nuclease and gRNA are encoded by a first vector and the partial ABCA4 coding sequence is provided by a second vector.

In one embodiment of the invention a vector system is provided comprising:
(a) a first construct comprising a payload sequence, wherein the payload is a nucleic acid encoding a ZFN; and
(b) a second construct comprising a payload sequence, wherein the payload sequence is a partial human ABCA4 nucleotide sequence.

In a preferred embodiment of the invention a vector system is provided comprising:
(a) a first construct comprising a payload sequence, wherein the payload is a nucleic acid encoding SEQ ID NO:7; and
(b) a second construct comprising a payload sequence, wherein the payload sequence is a partial human ABCA4 nucleotide sequence comprising SEQ ID NO:1, or a sequence having at least 90% sequence identity thereto wherein integration of the partial sequence into the genome of a subject restores the expression and/or function of ABCA4.

In a preferred embodiment of the invention a vector system is provided comprising:
(a) a first construct comprising a payload sequence, wherein the payload is a nucleic acid sequence comprising SEQ ID NO:8, or a sequence having at least 90% sequence identity thereto wherein the variant sequence encodes a functional ZFN; and
(b) a second construct comprising a payload sequence, wherein the payload sequence is a partial human ABCA4 nucleotide sequence comprising SEQ ID NO:1.

In a preferred embodiment of the invention a vector system is provided comprising:
(a) a first construct comprising a payload sequence, wherein the payload is a nucleic acid sequence consisting of SEQ ID NO:8; and
(b) a second construct comprising a payload sequence, wherein the payload sequence is a partial human ABCA4 nucleotide sequence consisting of SEQ ID NO:1.

In a preferred embodiment of the invention a vector system is provided comprising:
(a) a first vector comprising a first construct comprising a payload sequence, wherein the payload is a nucleic acid sequence consisting of SEQ ID NO:8; and
(b) a second vector comprising a second construct comprising a payload sequence, wherein the payload sequence is a partial human ABCA4 nucleotide sequence consisting of SEQ ID NO:1.

In one embodiment of the invention a vector system is provided comprising:
(a) a first construct comprising a payload sequence, wherein the payload is a nucleic acid encoding a SaCas9; and
(b) a second construct comprising a payload sequence, wherein the payload sequence is a partial human ABCA4 nucleotide sequence.

In a preferred embodiment of the invention a vector system is provided comprising:
(a) a first construct comprising a payload sequence, wherein the payload is a nucleic acid sequence comprising SEQ ID NO:4, or a sequence having at least 90% sequence identity thereto wherein the variant nucleic acid sequence encodes a functional SaCas9; and
(b) a second construct comprising a payload sequence, wherein the payload sequence is a partial human ABCA4 nucleotide sequence comprising SEQ ID NO:1.

In a preferred embodiment of the invention a vector system is provided comprising:
(a) a first construct comprising a payload sequence, wherein the payload is a nucleic acid sequence consisting of SEQ ID NO:4; and
(b) a second construct comprising a payload sequence, wherein the payload sequence is a partial human ABCA4 nucleotide sequence consisting of SEQ ID NO:1.

In a preferred embodiment of the invention a vector system is provided comprising:
(a) a first vector comprising a first construct comprising a payload sequence, wherein the payload is a nucleic acid sequence consisting of SEQ ID NO:4; and
(b) a second vector comprising a second construct comprising a payload sequence, wherein the payload sequence is a partial human ABCA4 nucleotide sequence consisting of SEQ ID NO:1.

In one embodiment of the invention a vector system is provided comprising:
(a) a first construct comprising:
   (i) a payload sequence, wherein the payload is a nucleic acid encoding a SaCas9; and
   (ii) a nucleic acid sequence encoding a gRNA containing a sequence complementary to SEQ ID NO: 2; and
(b) a second construct comprising a payload sequence, wherein the payload sequence is a partial human ABCA4 nucleotide sequence.

In one embodiment of the invention a vector system is provided comprising:
(a) a first construct comprising:
   (i) a payload sequence, wherein the payload is a nucleic acid encoding a SaCas9; and
   (ii) a nucleic acid sequence encoding a gRNA containing a sequence complementary to SEQ ID NO: 3; and
(b) a second construct comprising a payload sequence, wherein the payload sequence is a partial human ABCA4 nucleotide sequence.

In a preferred embodiment of the invention a vector system is provided comprising:
(a) a first construct comprising:
   (i) a payload sequence, wherein the payload is a nucleic acid sequence comprising SEQ ID NO:4, or a sequence having at least 90% sequence identity thereto wherein the variant sequence encodes a functional SaCas9; and
   (ii) a nucleic acid sequence encoding a gRNA containing a sequence complementary to SEQ ID NO: 2; and
(b) a second construct comprising a payload sequence, wherein the payload sequence is a partial human ABCA4 nucleotide sequence comprising SEQ ID NO:1.

In a preferred embodiment of the invention a vector system is provided comprising:
(a) a first construct comprising:
   (i) a payload sequence, wherein the payload is a nucleic acid sequence comprising SEQ ID NO:4, or a sequence having at least 90% sequence identity thereto wherein the variant sequence encodes a functional SaCas9; and
   (ii) a nucleic acid sequence encoding a gRNA containing a sequence complementary to SEQ ID NO: 3; and
(b) a second construct comprising a payload sequence, wherein the payload sequence is a partial human ABCA4 nucleotide sequence comprising SEQ ID NO:1.

In a preferred embodiment of the invention a vector system is provided comprising:
(a) a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 2; and
(b) a second construct comprising a payload sequence, wherein the payload sequence is a partial human ABCA4 nucleotide sequence consisting of SEQ ID NO:1.

In a preferred embodiment of the invention a vector system is provided comprising:
(a) a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 3; and
(b) a second construct comprising a payload sequence, wherein the payload sequence is a partial human ABCA4 nucleotide sequence consisting of SEQ ID NO:1.

In a preferred embodiment of the invention a vector system is provided comprising:
(a) a first vector comprising a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 2; and
(b) a second vector comprising a second construct comprising a payload sequence, wherein the payload sequence is a partial human ABCA4 nucleotide sequence consisting of SEQ ID NO:1.

In a preferred embodiment of the invention a vector system is provided comprising:
(a) a first vector comprising a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 3; and
(b) a second vector comprising a second construct comprising a payload sequence, wherein the payload sequence is a partial human ABCA4 nucleotide sequence consisting of SEQ ID NO:1.

In one embodiment of the invention a vector system is provided comprising:
(a) a first construct comprising:
   (i) a payload sequence, wherein the payload is a nucleic acid encoding a SaCas9; and
   (ii) a nucleic acid sequence encoding a gRNA containing a sequence complementary to SEQ ID NO: 2; and
(b) a second construct comprising:
   (i) a payload sequence, wherein the payload sequence is a partial human ABCA4 nucleotide sequence; and
   (ii) guide RNA target sequences inserted on either side of the payload sequence in inverted orientations.

In one embodiment of the invention a vector system is provided comprising:
(a) a first construct comprising:
   (i) a payload sequence, wherein the payload is a nucleic acid encoding a SaCas9; and
   (ii) a nucleic acid sequence encoding a gRNA containing a sequence complementary to SEQ ID NO: 3; and
(b) a second construct comprising:
   (i) a payload sequence, wherein the payload sequence is a partial human ABCA4 nucleotide sequence; and
   (ii) guide RNA target sequences inserted on either side of the payload sequence in inverted orientations.

In a preferred embodiment of the invention a vector system is provided comprising:
(a) a first construct comprising:
   (i) a payload sequence, wherein the payload is a nucleic acid sequence comprising SEQ ID NO:4, or a sequence having at least 90% sequence identity thereto wherein the variant sequence encodes a functional SaCas9; and
   (ii) a nucleic acid sequence encoding a gRNA containing a sequence complementary to SEQ ID NO: 2; and
(b) a second construct comprising:
   (i) a payload sequence, wherein the payload sequence is a partial human ABCA4 nucleotide sequence comprising SEQ ID NO:1; and
   (ii) guide RNA target sequences inserted on either side of the payload sequence in inverted orientations, said inverted guide RNA target sequences comprising or consisting of SEQ ID NO:5.

In a preferred embodiment of the invention a vector system is provided comprising:
(a) a first construct comprising:
   (i) a payload sequence, wherein the payload is a nucleic acid sequence comprising SEQ ID NO:4, or a sequence having at least 90% sequence identity thereto wherein the variant sequence encodes a functional SaCas9; and
   (ii) a nucleic acid sequence encoding a gRNA containing a sequence complementary to SEQ ID NO: 3; and
(b) a second construct comprising:
   (i) a payload sequence, wherein the payload sequence is a partial human ABCA4 nucleotide sequence comprising SEQ ID NO:1; and
   (ii) guide RNA target sequences inserted on either side of the payload sequence in inverted orientations, said inverted guide RNA target sequences comprising or consisting of SEQ ID NO:6.

In a preferred embodiment of the invention a vector system is provided comprising:
(a) a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 2; and
(b) a second construct comprising:
   (i) a payload sequence, wherein the payload sequence is a partial human ABCA4 nucleotide sequence comprising SEQ ID NO:1; and
   (ii) guide RNA target sequences inserted on either side of the payload sequence in inverted orientations, said inverted guide RNA target sequences comprising or consisting of SEQ ID NO:5.

In a preferred embodiment of the invention a vector system is provided comprising:
(a) a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 3; and
(b) a second construct comprising:
   (i) a payload sequence, wherein the payload sequence is a partial human ABCA4 nucleotide sequence comprising SEQ ID NO:1; and
   (ii) guide RNA target sequences inserted on either side of the payload sequence in inverted orientations, said inverted guide RNA target sequences comprising or consisting of SEQ ID NO:6.

In a preferred embodiment of the invention a vector system is provided comprising:
(a) a first vector comprising a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 2; and
(b) a second vector comprising a second construct comprising:
   (i) a payload sequence, wherein the payload sequence is a partial human ABCA4 nucleotide sequence consisting of SEQ ID NO:1; and
   (ii) guide RNA target sequences inserted on either side of the payload sequence in inverted orientations, said inverted guide RNA target sequences comprising or consisting of SEQ ID NO:5.

In a preferred embodiment of the invention a vector system is provided comprising:
(a) a first vector comprising a first construct comprising:
   (i) a payload sequence, wherein the payload is a nucleic acid sequence consisting of SEQ ID NO:4; and
   (ii) a nucleic acid sequence encoding a gRNA containing a sequence complementary to SEQ ID NO: 3; and
(b) a second vector comprising a second construct comprising:
   (i) a payload sequence, wherein the payload sequence is a partial human ABCA4 nucleotide sequence consisting of SEQ ID NO:1; and
   (ii) guide RNA target sequences inserted on either side of the payload sequence in inverted orientations, said inverted guide RNA target sequences comprising or consisting of SEQ ID NO:6.

The term “vector” refers to a nucleic acid molecule capable of transporting another nucleic acid. Non-limiting exemplary vectors include plasmids, phagemids, cosmids, artificial chromosomes, minichromosomes, transposons, viral vectors, and expression vectors. Viral vectors include, but are not limited to, adenovirus, lentivims, alphavims, enterovirus, pestivirus, baculovims, herpesvirus, Epstein Barr virus, papovavims, poxvirus, vaccinia vims, and herpes simplex vims. In a preferred embodiment, the vector is an adenoviral associated vector (AAV).

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, recently identified from primate brain. In vectors of the invention, the genome may be derived from any AAV serotype. The capsid may also be derived from any AAV serotype. The genome and the capsid may be derived from the same serotype or different serotypes. AAV vector serotypes can be matched to target cell types.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:
Clade A: AAV1 NC_002077, AF063497, AAV6 NC_001862, Hu. 48 AY530611, Hu 43 AY530606, Hu 44 AY530607, Hu 46 AY530609
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,
Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623
Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013
Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556
Clade F: Hu14 (AAV9) AY530579, Hu31 AY530596, Hu32 AY530597, Clonal Isolate AAV5 Y18065, AF085716, AAV 3 NC_001729, AAV 3B NC 001863, AAV4 NC_001829, Rh34 AY243001, Rh33 AY243002, Rh32 AY243003/

The preferred serotype of the vectors of the invention are AAV8, AAV9 and AAV5. The skilled person would be able to select further appropriate serotypes, clades, clones or isolates of AAV for use in the present invention on the basis of their common general knowledge.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al, 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

It should be understood however that the invention also encompasses use of an AAV genome of other serotypes that may not yet have been identified or characterised. The AAV serotype determines the tissue specificity of infection (or tropism) of an AAV virus.

Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (Virology Journal, 2007, 4:99), and in Choi et al and Wu et al, referenced above.

Derivatives of an AAV genome include any truncated or modified forms of an AAV genome which allow for expression of a Rep-1 transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs will preferably flank the expression construct cassette containing the promoter and transgene of the invention. The inclusion of one or more ITRs is preferred to aid packaging of the vector of the invention into viral particles. In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

With reference to the AAV2 genome, the following portions could therefore be removed in a derivative of the invention: One inverted terminal repeat (ITR) sequence, the replication (rep) and capsid (cap) genes. However, in some embodiments, including in vitro embodiments, derivatives may additionally include one or more rep and/or cap genes or other viral sequences of an AAV genome.

A derivative may be a chimeric, shuffled or capsid-modified derivative of one or more naturally occurring AAV viruses. The invention encompasses the provision of capsid protein sequences from different serotypes, clades, clones, or isolates of AAV within the same vector. The invention encompasses the packaging of the genome of one serotype into the capsid of another serotype i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population.

The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The properties of the constructs and vectors of the invention can be tested using techniques known by the person skilled in the art. In particular, a sequence of the invention can be assembled into a vector of the invention and delivered to a test animal, such as a mouse, and the effects observed and compared to a control.

### Pharmaceutical Compositions

The present disclosure includes pharmaceutical compositions comprising at least one of the first or second constructs of the invention or at least one of the first or second constructs of the invention incorporated into a vector.

Pharmaceutical compositions also include one or more of a pharmaceutically acceptable excipient, carrier or diluent. Exemplary pharmaceutically acceptable excipients such as carriers, solvents, stabilizers, adjuvants, diluents, etc., depending upon the particular mode of administration and dosage form. Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

Dosages and dosage regimes can be determined within the normal skill of the medical practitioner responsible for administration of the composition.

### Host Cells

Any suitable host cell can be used to produce the constructs or vectors of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

### Combination Therapy

The constructs, vectors and/or pharmaceutical compositions can be used in combination with any other therapy for the treatment of conditions caused by the mutation of ABCA4. In particular, constructs, vectors and/or pharmaceutical compositions can be used in combination with any other therapy for the treatment of Stargardt disease.

### Kits

The present disclosure provides kits for carrying out the methods described herein. A kit can include one or more of the first and second construct or vector of the invention. Components of a kit can be in separate containers, or combined in a single container.

Any kit described above can further comprise one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labelling of the container of the kit or components.

### Medical Use Thereof

The constructs, vectors and/or pharmaceutical compositions of the invention may be used in the treatment of a condition caused by a mutation in the ABCA4 gene. The treatment according to the present disclosure can ameliorate one or more symptoms associated with retinal dystrophy by increasing the amount of functional ABCA4 expressed in the retinal tissue of individual.

Described herein, the vector comprising a construct encoding a nuclease is for use in simultaneous, separate, or sequential combination with a vector comprising an expression construct comprising a partial human ABCA4 nucleotide sequence, for the treatment of a retinal dystrophy; optionally wherein the retinal dystrophy is Stargardt disease, cone dystrophy, cone-rod dystrophy, or retinitis pigmentosa, further optionally wherein the Stargardt disease is STGD1.

Also described herein is a vector comprising a construct comprising a partial human ABCA4 nucleotide sequence, for use in simultaneous, separate, or sequential combination with a vector comprising an expression construct encoding a nuclease, for the treatment of a retinal dystrophy; optionally wherein the retinal dystrophy is Stargardt disease, cone dystrophy, cone-rod dystrophy, or retinitis pigmentosa, further optionally wherein the Stargardt disease is STGD1.

The constructs, vectors and/or pharmaceutical compositions of the invention may be used in the treatment of amelioration of retinal dystrophies, such as Stargardt disease, cone dystrophy, cone-rod dystrophy, or retinitis pigmentosa, optionally wherein the Stargardt disease is STGD1. In a preferred embodiment, the constructs, vectors and/or pharmaceutical compositions of the invention may be used in the treatment or prevention, or amelioration, of Stargardt disease.

Described herein, the constructs, vectors and/or pharmaceutical compositions may be administered subretinally or by intravitreal injection. In a preferred embodiment, the constructs, vectors and/or pharmaceutical compositions are administered subretinally.

Described herein is the use of the first and second construct or vector of the invention in the manufacture of a medicament for the treatment or prevention of a retinal dystrophy, such as Stargardt disease. Also described is a method of treating or preventing a retinal dystrophy, such as Stargardt disease in a patient in need thereof comprising administering a therapeutically effective amount of a first and second construct or vector of the invention to the patient.

The dose of a vector of the invention may be determined according to various parameters, especially according to the age, weight and condition of the patient to be treated; the route of administration; and the required regimen. Again, a physician will be able to determine the required route of administration and dosage for any particular patient. For example, a suitable dose of a vector of the present invention may be in the range of 6.7×10¹³ vg/kg to 2.0×10¹⁴ vg/kg, where vg=viral genome.

The dose may be provided as a single dose, but may be repeated in cases where vector may not have targeted the correct region. The treatment is preferably a single permanent injection, but repeat injections, for example in future years and/or with different AAV serotypes may be considered.

### EXAMPLES

### Methods

CRISPR Cas9 sgRNA

Guide RNAs were designed using Benchling design tool (Benchling.com) and produced de novo by Sigma-Aldrich. Two candidate sgRNA sequences targeting intron 16 of ABCA4 were chosen based on in silico assessment by the design tool algorithm. The sgRNAs were tested for efficacy of ABCA4 genome editing by transfection of HEK293T cells with a single construct comprising two expression cassettes: EFS promoter-SaCas9 gene and either sgRNA driven by U6 promoter. After 72 h, the predicted insertion site was PCR amplified from genomic DNA (isolated using QuickExtract™ DNA Extraction Solution 1.0). Efficiency of insertion/deletion (INDEL) formation was analysed using the TIDE webtool (Brinkman et al, 2014 NARS).

Cutting efficiency of sgRNAs in photoreceptor cells was tested in human iPS cell derived retinal organoids. pAAV constructs were created carrying two expression cassettes: 1 for either of the sgRNAs and 1 for SaCas9. AAV serotypes 7m8 or ShH10 were used for transduction of retinal organoids. Cutting efficiencies were assessed by TIDE analysis as described above in whole EB lysates, and in purified photoreceptor and non-photoreceptor lysates after FACS sorting for CD73 (extracellular photoreceptor marker). The most efficient sgRNA construct was taken forward for further studies.

A separate set of sgRNAs specific for the mouse Abca4 intron 16 was designed and tested on mouse cells as described above to enable subsequent in vivo experiments in the mouse retina to test the concept of the therapy.

ABCA4^{exon17-50} construct

The truncated ABCA4 gene was designed on paper and produced de novo by Invitrogen. HITI cutting sites specific for the optimal sgRNA were included in the manufactured construct, with restriction sites that allowed them to be included or excluded from subsequent cloning steps. The construct was cloned w/ and w/o HITI into the pAAV backbone carrying the AAV2 ITRs for the production of AAV vectors.

The AAV-donor.eGFP constructs (w/ and w/o HITI) were produced by cloning a 2A-eGFP cassette in frame into exon 44 of the ABCA4^{exon17-50} coding sequence, replacing exons 44-50.

Once inserted into intron 16 of the ABCA4 genomic location, gene expression from the endogenous ABCA4 promoter would create a hybrid genomic-recombinant transcript, which will be processed normally. Splicing from the endogenous exon 16 to the exogenous exon 17 would create a full-length ABCA4 mRNA (for the Donor-ABCA4 constructs) for therapeutic applications, and a ABCA4*-eGFP fusion protein (for the Donor-eGFP constructs) for screening/visualisation applications. The 2A sequence between the ABCA4* and eGFP is a self-cleaving peptide that releases eGFP from the fusion protein to ensure fluorescence is not affected by the fusion.

Note that the exon16-17 boundary of the human and mouse ABCA4 genes is located at identical positions, so the human constructs were suitable for use in the mouse retina, albeit with a set of HITI sites specific for the mouse sgRNA. Integration into the mouse chromosome at the correct location would create a mouse-human hybrid gene.

### Production, Purification and Administration of AAV Vectors

Performed as described in Nishiguchi et al (2015, Nat Comms 6006). In vitro experiments were all performed using AAV serotype ShH10; in vivo experiments with AAV2/8.

Assessment of Targeted Integration into the ABCA4 Intron 16 Locus

Done in vivo in mouse retina and in vitro in human Stargardt patient iPS cell-derived retinal organoids:
Mouse Two AAV2/8 vectors, the first carrying the mouse Abca4 sgRNA and Cas9 cassettes, the second carrying the ABCA4^{exon17-44}-eGFP fusion gene, were mixed 1:1 and injected subretinally into C57BL6/J mice at a dose of 10¹⁰ viral genomes (total). After 1 month and 2 months, animals were killed and integration of ABCA4^{exon17-44}-eGFP into the retina was assessed by GFP fluorescence. Qualitative analysis was performed on retinal sections, counterstained for various retinal marker proteins. Quantitive assessment was performed by fluorescence analysis of single cells after dissociation of the retina (i.e. FACS w/o sorting the cells). Additional staining for CD73 was used to allow identification of photoreceptors and look specifically at genomic integration in photoreceptors.
Human Two AAV 7m8 vectors, the first carrying the human ABCA4 sgRNA and Cas9 cassettes, the second carrying the ABCA4^{exon}17-5° gene, were mixed 1:1 and 2×10¹² total viral genomes were added to single ABCA4-deficient organoids at 17-20 weeks of culture. Four weeks post administration, organoids were embedded and cryo-sections were prepared. Abcam antibody Ab77285, diluted 1: 100, was used to visualize ABCA4 protein. Staining for Recoverin, Rhodopsin and Crx was used to allow identification of photoreceptors at various stages of development.

### Guide RNAs

Guide RNAs were designed using Benchling design tool (Benchling.com). Two candidate sgRNA sequences targeting intron 16 of ABCA4 were chosen based on cutting efficiency predicted by the design tool algorithm. The target sequences (i.e. the sequence of intron 16 that is recognized by the guide RNA) are:

gRNAHu1:
TAAAGATCCAGACCTGCCCCGAGGAAT
gRNAHu2:
CTTATAAGGATACCAACTGGATTGGAT

The PAM site is underlined and the region which is complementary to the guide RNA is in bold.

The sgRNAs were tested for efficacy of ABCA4 genome editing by transfection of HEK293T cells with a single construct comprising two expression cassettes: EFS promoter-SaCas9 gene and either sgRNA driven by U6 promoter. After 72 h, the predicted insertion site was PCR amplified from genomic DNA. Efficiency of insertion/deletion (INDEL) formation as a measure of cutting activity was analysed using the TIDE webtool. Cutting efficiency of sgRNAs in photoreceptor cells was tested in human iPS cell derived retinal organoids. pAAV constructs were created carrying two expression cassettes: 1 for either of the sgRNAs and 1 for SaCas9. AAV serotypes 7m8 or ShH10 were used for transduction of retinal organoids. Cutting efficiencies were assessed by TIDE analysis as described above in whole EB lysates, and in purified photoreceptor and non-photoreceptor lysates after FACS sorting for CD73 (extracellular photoreceptor marker). The most efficient sgRNA (gRNAHu1) construct was taken forward for further studies.

### Example 1—Development of CRISPR/Cas9 that Cleaved the Human ABCA4 Gene

Classic gene replacement therapy for ABC4A is not feasible due to the large size of the ABCA4 coding sequence (6.8 kb). Instead, a system was developed where gene editing (CRISPR/Cas9 based) was used to create a double strand break in intron 17 of the ABCA4 gene. A second AAV vector was supplied which carried the coding sequence from exon 18 onward including a splice acceptor site. In a proportion of the cells, the endogenous DNA repair system will insert the AAV genome carrying the 3′ ABCA4 fragment into the genomic break, allowing splicing from the endogenous exon 17 to the transgenic exon 18-50. It was tested whether this system would be able to rescue ABCA4 expression and thus be able to prevent degeneration in patients who have at least 1 mutation 3′ to exon 17. Inclusion of CRISPR recognition sites in the AAV-ABCA4^{exon18-50} construct was expected to increase the efficiency of insertion of the correct vector genome in the correct orientation.

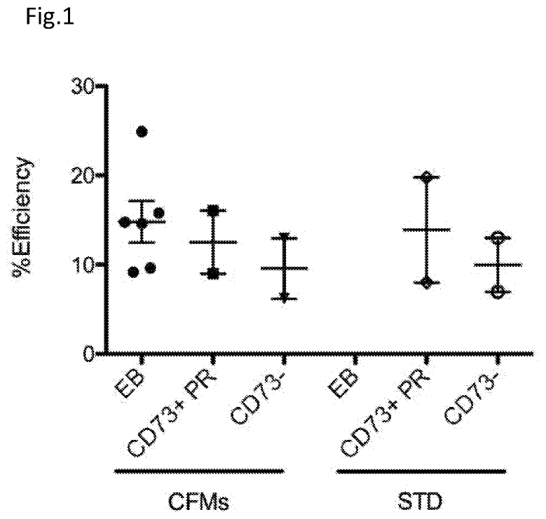

TIDE analysis of ABCA4 CRISPR/Cas9 delivered by AAV-SsH10 to human iPS cell derived retinal organoids (FIG. 1) indicated that double strand break formation at the cleavage site was present in 10-20% of genomes for whole EBs, photoreceptors only (CD73+) and remaining cells (CD73−). Note that the average transduction efficiency of AAV-SsH10 in human retinal organoids photoreceptors was ˜20%, suggesting >50% cutting efficiency of genomes in transduced cells.

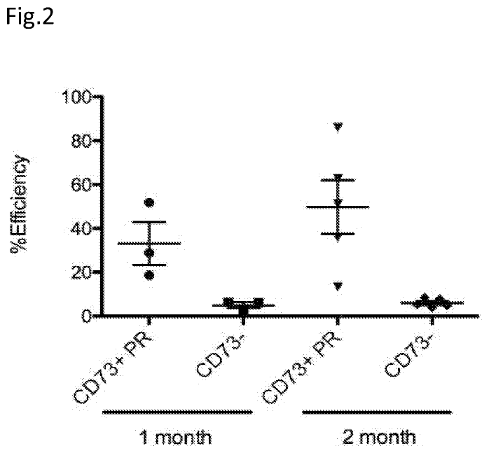

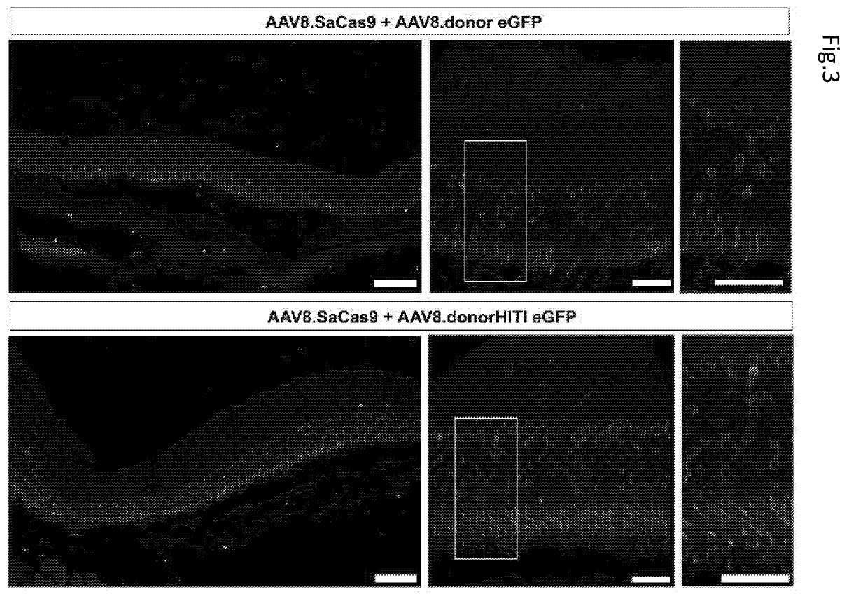

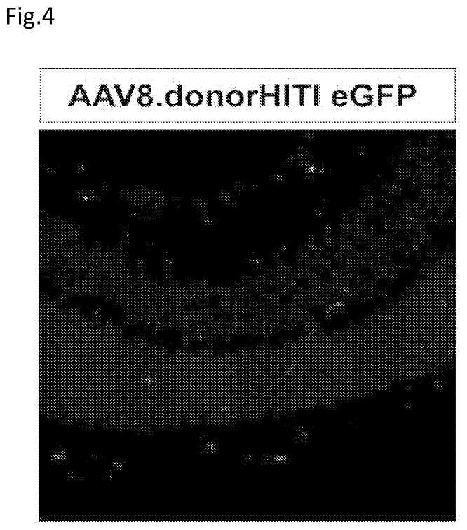

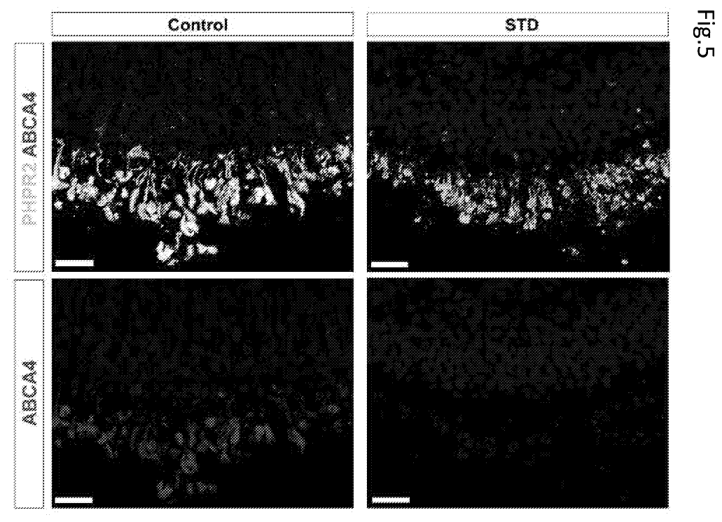

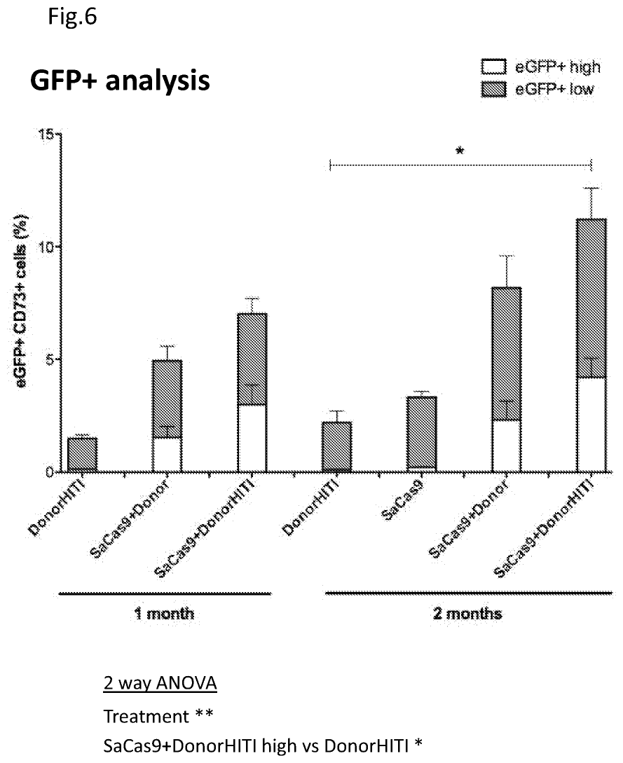

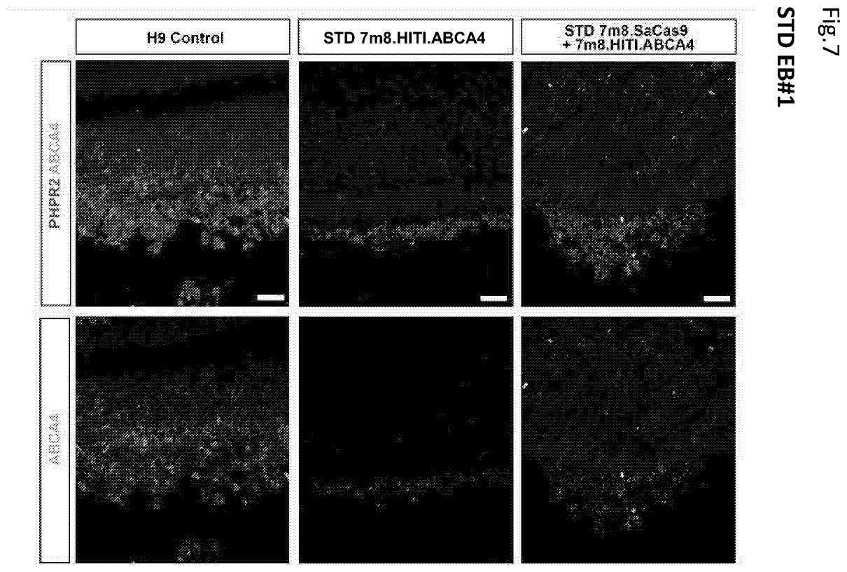

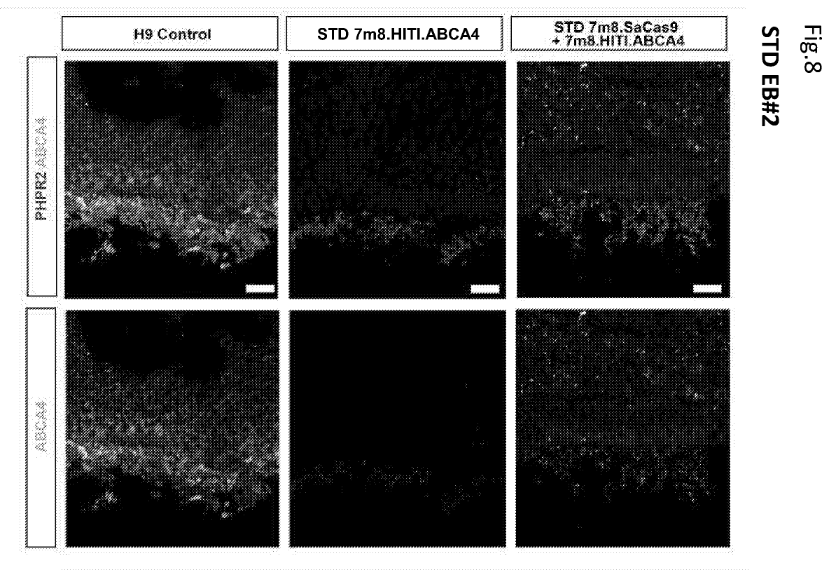

FIG. 2 shows delivery of mouse Abca4-specific CRiSPR/Cas9 in vivo. TIDE analysis showed that high efficiency double strand break formation is possible in CD73+ photoreceptors in vivo.

### Example 2—In Vivo Proof-of-Concept for AAV Insertion into Specific Double Strand Breaks

To show that it was possible to insert a transgenic ABCA4^{exon18-50} construct into ABCA4 intron 17 in photoreceptor cells in vivo, a fusion protein of ABCA4^{exon18-39} with GFP was produced. If inserted in the correct location in the mouse gene, the endogenous Abca4 expression would drive the partial human ABCA4 sequence and the GFP gene by splicing from the mouse exon 17 to the human exon 18, resulting in GFP protein.

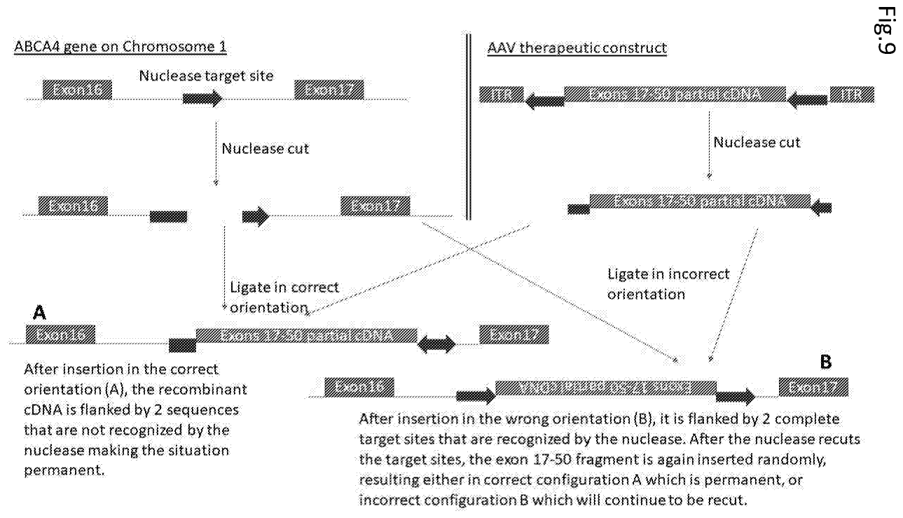

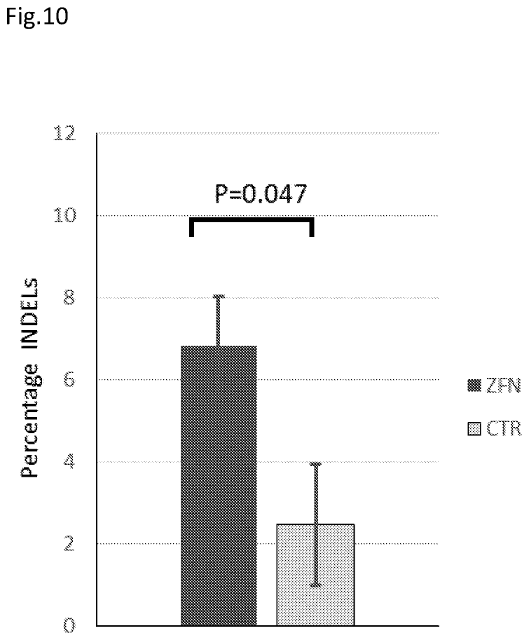

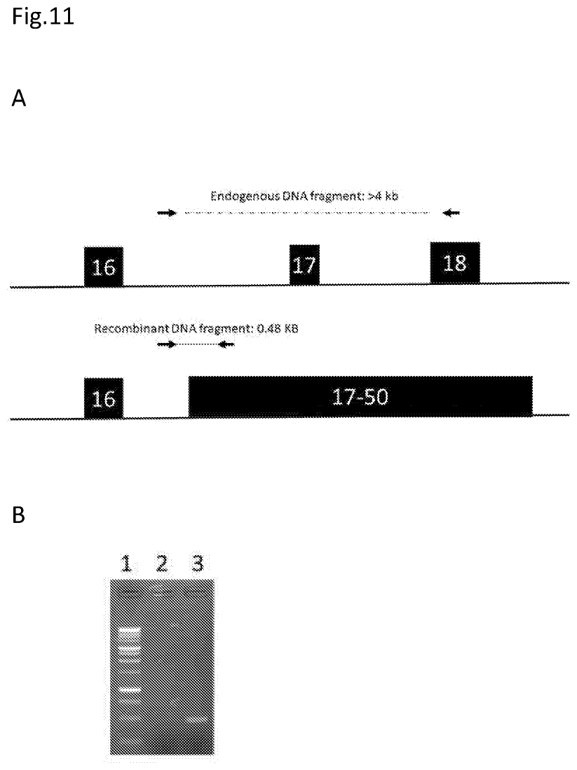

Using AAV carrying the mouse Abca4 CRISPR construct (AAV.SaCas9) in combination with AAV carrying the donor ABCA4-GFP fusion gene (AAV.donor eGFP), there was GFP protein in a minority of the cells (FIG. 3). The same experiment using an AAV-ABCA4-GFP genome flanked by “HITI” CRISPR recognition sites (AAV.donorHITI eGFP) resulted in a substantially greater number of cells expressing GFP, indicating that the inclusion of HITI sites is indeed supporting the correct insertion of the donor genome (FIG. 3). The result suggests that it may be feasible to correct enough cells to provide a therapeutic benefit.

FIG. 4 shows that injection of the HITI donor construct in the absence of the AAV-CRISPR/Cas9 vector did not result in cells expressing GFP, indicating that the GFP in FIG. 3 is not due to leaky expression from the AAV genome, or due to random insertion into the genome.

FIG. 5 shows that retinal organoids from Stargardt patient-derived iPS cells (STD) do not stain for the ABCA4 protein, but show otherwise normal morphology.

### Example 3—In Vivo Proof of Concept of Targeted Integration

Experiments were conducted to transduce retinal organoids with AAV-SaCas9 and AAV-HITI-ABCA4^{exon17-50}. Insertion of the donor ABCA4^{exon17-50} gene into the correct location was expected to restore ABCA4 signal in a subset of the cells.

FIG. 6 shows the percentage of GFP+ cells after subretinal injection in WT mice. Two populations of GFP+ cells were identified; strongly positive cells were absent in retinas transduced with only the ABCA4 donor vector or only the SaCas9 cutting vector. Strongly positive cells increased to 5% of total photoreceptors when using both vectors. Some weakly positive cells were present in retinas transduced with single vectors, but their numbers increased in double transduced retinas.

There was a trend for better treatment using Donor vector flanked by CRISPR recognition sites (Donor HITI) relative to Donor without cutting sites (Donor).

### Example 4—Targeted Integration in ABCA4-STD Organoids

FIG. 7 shows that ABCA4−/− human retinal organoids (STD) which were transduced with SaCas9 vector and DonorHITI vector (right) produced greater amounts of ABCA4 protein (white) than organoids transduced with DonorHITI vector only (middle). Wildtype organoids (H9) were provided as a positive control.

This data shows that only in the presence of the Cas9 cutting vector does the Donor vector integrate leading to full length protein. FIG. 8 shows an independent set of organoids treated identically to the previous slide, showing the same results.

### Example 5—Targeted Integration of ABCA4^{exon17-50} Using a Zinc Finger Nuclease

### Zinc Finger Nuclease: Cutting Efficiency

A zinc finger nuclease (ZFN) targeting intron 16 of ABCA4 was designed de novo from information available in the literature (https://www.nature.com/articles/nprot.2006.231/tables/1). Constructs carrying a zinc finger nuclease expression cassette with the CMV promoter were produced commercially (Genscript Biotech, Netherlands).

Zinc finger plasmids were transfected into 293T cells in 4 wells of 6-well plate (2 μg of DNA per well) using PEI (5 μg per well) in 400 μL of DMEM without additives for 4 hours. A further 4 wells were left non-transfected to function as negative controls. After incubation for 36 hours, cells were harvested and genomic DNA was isolated using a Blood and Tissue DNA kit (Qiagen, UK) and eluted in 100 μL. The area around the cutting site was amplified using Phusion high-fidelity DNA polymerase (Thermo-Fisher, UK), with 2 μL template DNA in a 60 μL volume. The following amplification primers were used:

Forward:
GAAAGGAAACAGAGGCACAC
Reverse:
AGATAAAGATCCAGACCTGCC.

PCR fragments were assessed by gel electrophoresis, before commercial sequencing (Genewiz, UK). Successful sequences were obtained from 4 test samples and 3 control samples. Efficiency of insertion/deletion (INDEL) formation was analysed using the TIDE webtool (Brinkman et al, 2014 NARS) (FIG. 10).

Efficiency of INDEL creation 40 hours after transfection of 293T cells with a plasmid expressing zinc finger nuclease ZFN16C, targeting intron 16 of the human ABCA4 gene was analysed (FIG. 10). TIDE assessment of sequencing traces showed that there was a significantly greater number of INDELs found when comparing zinc finger nuclease treated cells against control cells (ZNF) than when comparing control cells against each other. The relatively modest levels of INDEL formation may be the result of the short incubation times. Moreover, zinc finger nucleases created single-stranded DNA overhangs during the cleaving process, which encourages perfect repair of the DSB, even during non-homologous end-joining, which goes undetected as a cleaving event in the TIDE analysis.

### Zinc Finger Nuclease: ABCA4 Template Insertion

Zinc finger plasmids were transfected into 293T cells in 1 well of 6-well plate (2 μg of DNA per well) using PEI (5 μg per well) in 400 μL of DMEM without additives for 4 hours. A further well was left non-transfected to function as negative control. After incubation for 36 hours, AAV-SsH10 vector carrying the ABCA4¹⁷⁻⁵⁰ construct was added to both wells. After 7 days, cells were harvested and genomic DNA was isolated using a Blood and Tissue DNA kit (Qiagen, UK) and eluted in 100 μL. Insertion of ABCA4¹⁷⁻⁵⁰ into intron 16 of the ABCA4 genomic site was assessed by PCR amplification using GoTaq DNA polymerase (Promega, UK), with 1 μL template DNA in a 20 μL volume. The forward primer annealed upstream of the zinc finger cutting site and a reverse primer in intron 18. Due to the presence of part of intron 16 and all of intron 17 (>4 kb) amplification of the endogenous genomic sequence is not possible (see FIG. 11A). The following amplification primers were used:

Forward:
AGAAAGGAAACAGAGGCACAC
Reverse:
TTCACGCATACCCCAGGAAC

Presence of a 0.48 bp insert was assessed by gel electrophoresis (FIG. 11B).

PCR amplification of ABCA4¹⁷⁻⁵⁰ inserted into intron 16 of the endogenous ABCA4 gene. The forward primer anneals to intron 16, the reverse primer to exon 18. Amplification of the >4 kb fragment of the endogenous gene is unfeasible using an PCR extension time of 40 seconds. The fragment amplified from the inserted recombinant coding sequence is 0.48 kb. Presence of amplification fragments was assessed by gel electrophoresis. Presence of a 0.48 kb band indicated that there was integration of the recombinant ABCA4¹⁷⁻⁵⁰ into the genomic locus only in the presence of ZFN16C.

1. A vector system comprising:
(a) a first construct comprising a payload sequence, wherein the payload sequence is a nucleic acid encoding a nuclease; and
(b) a second construct comprising a payload sequence, wherein the payload sequence is a partial human ABCA4 nucleotide sequence.

2. The vector system of claim 1, wherein a first vector comprises the first construct and a second vector comprises the second construct.

3. The vector system of claim 1, wherein:
(a) the partial human ABCA4 sequence comprises a partial intron and a cDNA encoding the wildtype ABCA4 sequence downstream from said intron; or
(b) the partial human ABCA4 sequence comprises one or more of exons 17 to 50.

4-6. (canceled)

7. The vector system of claim 1, wherein the nuclease is a CRISPR nuclease, a transcription activator-like effector nuclease (TALEN) or a Zinc Finger Nuclease (ZFN).

8. The vector system of claim 7, wherein:
(a) the first construct encodes a CRISPR nuclease selected from Cas9, Cpfl, Casl2b (C2cl), Casl3a (C2c2), Casl3b (C2c6), and C2c3, optionally wherein the Cas9 is SaCas9; or
(b) the first construct encodes a CRISPR nuclease and additionally comprises a nucleic acid sequence encoding a guide RNA (gRNA) comprising a sequence that is complementary to a target sequence within intron 16 of the endogenous human ABCA4 gene.

9. (canceled)

10. The vector system of claim 89, wherein:
(a) the gRNA is complementary to SEQ ID NO: 2 or 3; or
(b) the second construct comprising a partial human ABCA4 nucleotide sequence is flanked by two inverted DNA sequences that are identical to the target DNA sequence.

11. (canceled)

12. The vector system of claim 10, wherein the second construct comprises in a 5′ to 3′ direction:
(a) i) SEQ ID NO: 5;
ii) SEQ ID NO: 1, or a sequence having at least 90% sequence identity thereto; and
iii) SEQ ID NO: 5; or
(b) i) SEQ ID NO:6;
ii) SEQ ID NO: 1, or a sequence having at least 90% sequence identity thereto; and
iii) SEQ ID NO:6.

13. (canceled)

14. The vector system of claim 8, wherein the gRNA is a single guide RNA molecule or a 2-piece guide RNA, optionally wherein the 2-piece guide RNA comprises a CRISPR RNA (crRNA-like) molecule and a trans-activating CRISPR RNA (tracrRNA-like) molecule.

15. The vector system of claim 7, wherein:
(a) the nuclease encoded by the nucleic acid sequence is a ZFN of SEQ ID NO: 7, or a sequence having at least 90% sequence identity thereto; or
(b) the ZFN is encoded by a nucleic acid sequence comprising SEQ ID NO:8, or a sequence having at least 90% sequence identity thereto.

16. (canceled)

17. The vector system of claim 1, wherein the payload sequences encoding the nuclease, and optionally the gRNAs, are operably linked to a ubiquitous promoter.

18. The vector system of claim 1, wherein the first and/or second vector is a viral vector; optionally
wherein the first and/or second viral vector is selected from a lentivirus, a retrovirus, an adenovirus, and an adeno-associated virus.

19. The vector system of claim 18, wherein the first and/or second vector is an adeno-associated virus (AAV) vector or comprises an AAV genome or a derivative thereof, optionally wherein:
said derivative is a chimeric, shuffled or capsid modified derivative;
said AAV genome is from a naturally derived serotype or isolate or clade of AAV; and/or
the AAV vector is selected from AAV8, AAV9 or AAV5.

20. A pharmaceutical composition comprising the vector system of claim 1.

21-24. (canceled)

25. A method of treating a retinal dystrophy, the method comprising administering the vector system as defined in claim 1 to a subject,
optionally wherein the retinal dystrophy is Stargardt disease, cone dystrophy, cone-rod dystrophy, or retinitis pigmentosa,
further optionally wherein the Stargardt disease is STGD1.

26. (canceled)

27. A vector comprising a construct:
(a) encoding a nuclease, for use in simultaneous, separate, or sequential combination with a vector comprising a construct comprising a partial human ABCA4 nucleotide sequence, for the treatment of a retinal dystrophy;
optionally wherein the retinal dystrophy is Stargardt disease, cone dystrophy, cone-rod dystrophy, or retinitis pigmentosa,
further optionally wherein the Stargardt disease is STGD1; or
(b) comprising a partial human ABCA4 nucleotide sequence, for use in simultaneous, separate, or sequential combination with a vector comprising a construct encoding a nuclease, for the treatment of a retinal dystrophy:
optionally wherein the retinal dystrophy is Stargardt disease, cone dystrophy, cone-rod dystrophy, or retinitis pigmentosa,
further optionally wherein the Stargardt disease is STGD1.

28. (canceled)

29. A method of treating a retinal dystrophy, the method comprising administering the pharmaceutical composition as defined in claim 20 to a subject,
optionally wherein the retinal dystrophy is Stargardt disease, cone dystrophy, cone-rod dystrophy, or retinitis pigmentosa,
further optionally wherein the Stargardt disease is STGD1.

30. The method of claim 25, wherein:
(a) the system is used to splice an endogenous partial ABCA4 sequence to an exogenous partial ABCA4 sequence to correct mutations in the downstream portion of the endogenous ABCA4 gene; or
(b) the system corrects one or more mutations in the ABCA4 gene of a non-dividing cell, optionally wherein the non-dividing cell is a retinal cell, further optionally wherein the cell is a photoreceptor cell, such as a rod photoreceptor cell or a cone photoreceptor cell; or
(c) (i) the retinal dystrophy is Stargardt disease, cone dystrophy, cone-rod dystrophy, or retinitis pigmentosa, optionally wherein the Stargardt disease is STGD1; or
(ii) the first and/or second vectors are administered subretinally or by intravitreal injection.
